(19) United States
(12) Patent Application Publication
(10) Pub. No.: US 2018/0360964 A1
(43) Pub. Date: Dec. 20, 2018

(54) PHARMACEUTICAL COMPOSITION AND A METHOD FOR PRODUCING THEREOF

(71) Applicant: MONASH UNIVERSITY MALAYSIA, Subang Jaya (MY)

(72) Inventor: MD. EZHARUL HOQUE CHOWDHURY, SUBANG JAYA (MY)

(21) Appl. No.: 16/008,051

(22) Filed: Jun. 14, 2018

(30) Foreign Application Priority Data
Jun. 14, 2017 (MY) PI 2017702177

(51) Int. Cl. A61K 47/02; C12N 15/113; A61K 31/7088; A61K 31/675; A61K 31/519; A61K 31/513; A61K 9/19

(52) U.S. Cl. CPC A61K 47/02; C12N 15/113; A61K 31/7088; C12N 2310/14; A61K 31/519; A61K 31/513; A61K 9/19; A61K 31/675

(57) ABSTRACT

The present invention provides a pharmaceutical composition, the pharmaceutical composition comprising a pharmaceutically active substance, an apatite-based matrix, and a surface modifying agent. Further, the apatite-based matrix comprises calcium ion, phosphate ion, hydrogen carbonate ion, magnesium ion and iron ion. Also, the surface modifying agent comprises a protein, a polymer or a combination thereof. Further, a method of producing the pharmaceutical composition (200) is disclosed.

### CROSS-REFERENCE TO RELATED APPLICATION

This application claims priority to Malaysia patent application no. PI 2017702177 filed on Jun. 14, 2017; the disclosure of which is incorporated by reference in its entirety.

### FIELD OF THE INVENTION

The present invention relates to the field of medical treatment, particularly a pharmaceutical composition comprising inorganic and organic components and a method of producing thereof. More particularly, the pharmaceutical composition comprising an inorganic apatite-based matrix and an organic surface modifying agent.

### BACKGROUND OF THE INVENTION

Extensive research efforts on developing an ideal drug delivery system constantly make progress and gradually improve the prospects of therapeutics as there are still many human diseases with high unmet medical needs. Subsequently, numerous drug carriers and drug targeting systems were developed and yet most of them are incapable to overcome several limitations such as drug degradation and loss, non-specific bio-distribution, low drug bioavailability, potential toxicity or side effects and low water solubility.

Advances in nanotechnology has a significant impact to conventional drug delivery system, for instance, biocompatible nanoscale drug carriers such as viral vectors, liposomes and polymeric nanoparticles have shown great promise to achieve more efficient and safer delivery of a myriad of therapeutics. While viral vectors have emerged as safe and effective delivery vehicles for gene therapy (Annu. Rev. Biomed. Eng. 2015, 17, 63-89), their practical use in clinical practices are restricted because of their immunogenicity and cytotoxicity from the clinical perspective. On the other hand, non-viral vectors such as lipid-mediated vectors and polymeric nanoparticles possess important safety advantage due to their reduced pathogenicity, low cost and ease of production. However, the main hurdle is their efficacy of delivery, which is relatively low when compared to that of viral vectors (Journal of Clinical and Diagnostic Research: JCDR 9.1 (2015): GE01-GE06).

Furthermore, the size of a drug carrier seems to be one of the important factors in determining the success of transporting and subsequently releasing the drug into the target cells. Larger particles are possibly to be cleared from the human body by phagocytosis, which is one of the body's innate modes of defense against invading pathogens and other particles (Djaldetti S H, Bergman M, Djaldetti R, Bessler H. Phagocytosis—the mighty weapon of the silent warriors. Microsc Res Tech. 2002, 57, 421-431), whereas small particles could be homogenously distributed throughout the body and rapidly undergo renal clearance upon intravenous administration (Choi, H. S. et al. Renal clearance of quantum dots. Nat. Biotechnol. 2007, 25, 1165-1170). Both of these processes are undesired for most drug delivery system and hence it is important to regulate the size and even the surface charge of the drug carrier in order to preclude their efficient clearance from the body.

It is increasingly known that apatite plays a crucial role in the medical application due to its biocompatibility and bioactivity. Since apatite exhibited promising result in drug delivery application, there are a number of solutions developed for producing an efficient pharmaceutical composition comprising inorganic and organic components that shows potential in tumour treatment and few of them have been discussed in following exemplary.

U.S. Pat. No. 9,295,640B2 describes a pharmaceutical composition that can produce a high antitumor effect by efficiently delivering a drug with antitumor activity to tumor tissues. The pharmaceutical composition comprises carbonate apatite nanoparticles containing a drug with antitumor activity and a pharmacologically acceptable solvent in which the nanoparticles are dispersed. The carbonate apatite nanoparticles containing the drug is subjected to an ultrasonic treatment. Further, albumin is added to further reduce the particle size and suppress the aggregation of the particles. While, the step of ultrasonic treatment may help reducing the size of particles, the drug could be significantly dissociated or released from the nanoparticle or even degraded by ultrasonic waves.

JP2011010549 discloses an organic-inorganic hybrid nanoparticle comprising a conjugate of a nucleic acid and a polyethylene glycol chain bound covalently to the nucleic acid and a calcium ion and a phosphate ion. The nucleic acid may be single strand to double strand oligo to polynucleotide, and can be selected from the group consisting of siRNA and DNA, or RNA aptamers. The nanoparticle has the limitation that it can be incorporated with nucleic-acid based drug only and the specificity on how the nucleic acid can be transported into the target cells by the nanoparticle still remain questionable. Moreover, since these particles are based on calcium phosphate or hydroxyapatite-based particles, the solubility or dissolution of the particles would be lower in endosomal acidic environment, limiting the release of drugs from the particles inside the cells. This could limit the efficiency of the drugs as they are only able to exhibit their effects when they are completely released from the particles.

Accordingly, there remains a need in the prior art to have an improved pharmaceutical composition which is flexible in regulating its size, surface modification and pH sensitivity in order to improve therapeutic efficacy and reduce of off-target effects. Further, the use of the prior arts in clinical practice has so far met only with a very limited success due to their incapability to bind with a myriad of drugs and also to release the drugs in sufficient amount into the target cells. Therefore, there is a need to have an improved pharmaceutical composition comprising inorganic and organic components and the method of producing thereof which overcomes the aforesaid problems and shortcomings.

### SUMMARY OF THE INVENTION

Embodiments of the present invention aim to provide a pharmaceutical composition comprising inorganic and organic components and a method of producing thereof. The inorganic component is an apatite-based matrix and the organic component is a surface modifying agent. The invention allows the size of pharmaceutical composition to be regulated in delicate manner in order to facilitate the uptake of drugs by the target cells and improve drug accumulation in each organ. In addition, the invention may confer a favourable pharmacokinetics and efficient release of drugs in the target cells through surface modification and pH sensitivity control on the pharmaceutical composition. Further, the invention has the capability of overcoming the limitation of poor complexation with multiple hydrophobic and hydrophilic drugs that encountered by the existing arts. Moreover, the invention is able to eliminate particles aggregation possibly caused by ionic and hydrophobic interactions among the apatite-based matrix, solvent and drug molecules.

In accordance with an embodiment of the present invention, a pharmaceutical composition comprises a pharmaceutically active substance, an apatite-based matrix and a surface modifying agent. Further, the apatite-based matrix comprises calcium ion, phosphate ion, hydrogen carbonate ion, magnesium ion and iron ion. Further, the surface modifying agent comprises a protein, a polymer or a combination thereof.

In accordance with an embodiment of the present invention, the apatitie-based matrix may further comprise at least one ion selected from strontium ion, fluoride ion, and barium ion or any combination thereof. In another preferred embodiment of the present invention, the apatite-based matrix may further comprises at least one carboxyl group-containing molecule selected from citrate, succinate, pyruvate, lactate, alpha-ketoglutarate, oxaloacetate, fumarate and malate. In still another embodiment of the present invention, the apatite-based matrix may comprise at least one ion and at least one carboxyl group-containing molecule.

In accordance with an embodiment of the present invention, the protein is streptavidin, transferrin, fibronectin, collagen, albumin, lactoferrin asialofetuin, lipoprotein or proteoglycan.

In accordance with an embodiment of the present invention, the polymer is polyethylene glycol (PEG). Further, each PEG is associated with a biotin moiety.

In accordance with an embodiment of the present invention, the size of the pharmaceutical composition is 5-999 nanometer.

In accordance with an embodiment of the present invention, the pharmaceutically active substance is selected from the group consisting of drug, protein, nucleic acid and any combination thereof.

In accordance with an embodiment of the present invention, the drug is an anti-tumour agent. Further, the anti-tumour agent is selected from the group comprising of antimetabolites, alkylating agents and antibiotics.

In accordance with an embodiment of the present invention, the nucleic acid is deoxyribonucleic acid (DNA), ribonucleic acid (RNA), oligonucleotide or polynucleotide.

In accordance with an embodiment of the present invention, the ribonucleic acid is siRNA, miRNA or antisense of RNA.

In accordance with an embodiment of the present invention, a method for producing the pharmaceutical composition comprises the steps of preparing a first mixture containing a pharmaceutically active substance and an apatite-based matrix, subjecting the first mixture to a first incubation, adding a surface modifying agent into the first mixture to form a second mixture, and subjecting the second mixture to a second incubation to form the pharmaceutical composition.

In accordance with an embodiment of the present invention, the first mixture is further added with at least one ion selected from strontium ion, fluoride ion and barium ion, or at least one carboxyl group-containing molecule selected from citrate, succinate, pyruvate, lactate, alpha-ketoglutarate, oxaloacetate, fumarate and malate, or any combination thereof before the first incubation step.

In accordance with an embodiment of the present invention, the first mixture is further added with a protein-based surface modifying agent before the first incubation step.

In accordance with an embodiment of the present invention, the apatite-based matrix is prepared by the steps comprising of preparing a first solution that contains calcium ion, adding the first solution into a second solution that contains phosphate ion, hydrogen carbonate ion, magnesium ion and iron ion.

In accordance with an embodiment of the present invention, the apatite-based matrix is prepared by the steps comprising of preparing a first solution that contains phosphate ion, adding the first solution into a second solution that contains calcium ion, hydrogen carbonate ion, magnesium ion and iron ion.

In accordance with an embodiment of the present invention, the second solution further comprising sodium chloride and glucose.

In accordance with an embodiment of the present invention, the concentration of sodium chloride is in a range of 10-1000 millimolar of the second solution.

In accordance with an embodiment of the present invention, the concentration of glucose is in a range of 10-1000 millimolar of the second solution.

In accordance with an embodiment of the present invention, the calcium ion concentration is in a range of 1-100 millimolar.

In accordance with an embodiment of the present invention, the phosphate ion concentration is in a range of 0.1-100 millimolar.

In accordance with an embodiment of the present invention, the hydrogen carbonate ion concentration is in a range of 10-100 millimolar.

In accordance with an embodiment of the present invention, the magnesium ion concentration is in a range of 1-100 millimolar.

In accordance with an embodiment of the present invention, the iron ion concentration is in a range of 1-100 millimolar.

In accordance with an embodiment of the present invention, the first mixture has a pH of 6.0-8.0.

In accordance with an embodiment of the present invention, each incubation is carried out at a temperature in a range of 25° C.-65° C.

In accordance with an embodiment of the present invention, the pharmaceutical composition is dispersed in a pharmacologically acceptable solvent when in use.

In accordance with an embodiment of the present invention, the pharmacologically acceptable solvent is a buffered cell culture medium solution or saline solution.

In accordance with an embodiment of the present invention, the pharmaceutical composition is subjected to lyophilisation to obtain a powder form.

In accordance with an embodiment of the present invention, the pharmaceutical composition is subjected to high pressure condensation to obtain a solid dosage form.

### BRIEF DESCRIPTION OF THE DRAWINGS

So that the manner in which the above recited features of the present invention can be understood in detail, a more particular description of the invention, briefly summarized above, may have been referred by embodiments, some of which are illustrated in the appended drawings. It is to be noted, however, that the appended drawing illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein:

FIG. 1 is a flow chart illustrating a formation process of a pharmaceutical composition in accordance with an embodiment of the present invention.

FIG. 2 illustrates a method of producing a pharmaceutical composition in accordance with an embodiment of the present invention.

FIG. 3 illustrates an infrared spectra of generated apatite-based matrix.

FIG. 4 illustrates a X-ray diffraction (XRD) pattern of generated apatite-based matrix.

FIG. 5 illustrates a X-ray fluorescence (XRF) pattern of generated apatite-based matrix.

FIG. 6 show the results for: (a) turbidity assessment by absorbance at 320 nm and (b) size measurement for the generated apatite-based matrix in an increasing concentration of exogenous calcium chloride (CaCl₂).

FIG. 7 shows the turbidity measurement by absorbance at 320 nm for the generated apatite-based matrix at different pH adjusted by adding 1N hydrochloric acid (HCl).

FIG. 8 illustrates the fluorescence intensity measurement of bound siRNA to the apatite-based matrix in percentage at different siRNA concentration when the siRNA concentration is increased.

FIG. 9 illustrate the diameter measurement of the apatite-based matrix, (a) loaded with and (b) without doxorubicin (Dox) and cyclophosphamide (Cyp) respectively.

FIG. 10 illustrates the fluorescence intensity measurement of bound siRNA to apatite-based matrix in percentage loaded with doxorubicin (Dox) when the concentration of Dox is increased.

FIGS. 11 (a) and (b) illustrate the fluorescence intensity measurement in relative light units (RLU) per mg protein of transfection of MCF-7 and 4T1 cells with luciferase plasmid-carrying apatite-based matrix.

FIG. 12 illustrate the results of diameter measurement of generated apatite-based matrix with different concentration of: (a) exogenous calcium chloride (CaCl₂), and (b) strontium chloride (SrCl₂) while other salts being constant.

FIG. 13 illustrates the tumour regression study on 4T1 induced tumour mouse model demonstrating changes in relative tumour outgrowth volume of mice (mm³) intravenously treated with (a) apatite-based matrix (CA); (b) free cyclophosphamide (Free Cyp); and (c) apatite-cyclophosphamide complex (CA-Cyp) respectively.

FIG. 14 illustrates the fluorescence intensity measurement of bio-distribution of pharmaceutical composition comprising apatite-based matrix incorporated with AF 488 siRNA with fibronectin and transferrin coating in brain, kidney, liver, lung, spleen and tumour of a tumour-bearing mouse model following intravenous injection of the pharmaceutical composition.

FIG. 15 illustrates the result of silver-stained SDS PAGE examination of the presence of the surface modifying agent on the apatite-based matrix. Surface modifying agent included biotinylated PEG, streptavidin and fibronectin. Lane 1, Streptavidin (control); Lane 2, Biotinylated PEG (control); Lane 3, Fibronectin (control); Lane 4 and 5, biotinylated-PEG apatite-based matrix with streptavidin; Lane 6 and 9, biotinylated PEG apatite-based matrix with streptavidin and further coated with fibronectin; Lane 7 and 8, apatite-based matrix (control).

FIG. 16 illustrates the result of BlueBANDit-stained SDS PAGE examination of the presence of serum protein (Fetal Bovine Serum) on the surface modified apatite-based matrix. Lane 1, FBS (control); Lane 2, Surface-modified apatite-based matrix with streptavidin (5 uL) and biotinylated PEG (5 uL) treated with FBS; Lane 3, Surface-modified apatite-based matrix with biotinylated PEG (5 uL) treated with FBS; Lane 4, apatite-based matrix treated with FBS; Lane 5, Apatite-based matrix (control); Lane 6, Surface-modified apatite-based matrix with streptavidin (2 uL) and biotinylated PEG (2 uL) treated with FBS; Lane 7, Surface-modified apatite-based matrix with biotinylated PEG (2 uL) treated with FBS.

FIG. 17 (a) to (c) illustrate the size measurement of pharmaceutical composition (surface modified, drug loaded apatite-based matrix). Apatite-based matrix alone (CA) and drug-loaded apatite-based matrix (CA+drug) were used as control. Surface modifications included streptavidin (+strep), streptavidin and biotinylated-PEG (+strep+PEG), and a combination of streptavidin, biotinylated-PEG and fibronectin (+strep+PEG+fib).

FIG. 18 (a) to (c) illustrate the zeta potential measurement of pharmaceutical composition (surface modified, drug loaded apatite-based matrix). Apatite-based matrix alone (CA) and drug-loaded apatite-based matrix (CA+drug) were used as control. Surface modifications included streptavidin (+strep), streptavidin and biotinylated-PEG (+strep+PEG), and a combination of streptavidin, biotinylated-PEG and fibronectin (+strep+PEG+fib).

FIG. 19 illustrates the tumour regression study demonstrating changes in relative tumour outgrowth volume (mm³) on a 4T1 induced breast tumour mouse model intravenously treated with i) apatite-based matrix as control (CA-treated); ii) apatite-based matrix incorporated with ESR1 siRNA (CA+ESR1); iii) apatite-based matrix incorporated with BCL-2 siRNA (CA+BCL-2); iv) apatite-based matrix incorporated with ESR1 and BCL-2 siRNAs (CA+ESR1+BCL-2); and v) without treatment (untreated) respectively.

FIG. 20 illustrates the tumour regression study demonstrating changes in tumour outgrowth volume (mm³) on a 4T1 induced breast tumour mouse model intravenously treated with i) apatite-based matrix as control (CA-treated); ii) apatite-based matrix incorporated with ERBB2 siRNA (CA+ERBB2); iii) apatite-based matrix incorporated with ESR1 siRNA (CA+ESR1); iv) apatite-based matrix incorporated with EGFR siRNA (CA+EGFR); v) apatite-based matrix incorporated with ESR1, ERBB2 and EGFR siRNAs (CA+ESR1+ERBB2+EGFR); and vi) without treatment (untreated) respectively.

FIG. 21 illustrates the tumour regression study demonstrating changes in relative tumour outgrowth volume (mm³) using a 4T1 induced breast tumour mouse model intravenously treated with i) apatite-based matrix as control (CA-treated); ii) apatite-based matrix incorporated with ROS1 siRNA (CA+ROS1); iii) apatite-based matrix incorporated with SHC1 siRNA (CA+SHC1); iv) apatite-based matrix incorporated with ROS1 and SHC1 siRNAs (CA+ROS1+SHC1); and v) without treatment (untreated) respectively.

FIG. 22 illustrates the tumour regression study demonstrating changes in relative tumour outgrowth volume (mm³) using a 4T1 induced breast tumour mouse model intravenously treated with i) apatite-based matrix as control (CA-treated); ii) apatite-based matrix incorporated with ROCK2 siRNA (CA+ROCK2); iii) apatite-based matrix incorporated with CAMK4 siRNA (CA+CAMK4); iv) apatite-based matrix incorporated with NFATC4 siRNA (CA+NFATC4); v) apatite-based matrix incorporated with RYR3 siRNAs (CA+RYR3); vi) apatite-based matrix incorporated with ROCK2, CAMK4, NFATC4 and RYR3 siRNAs (CA+ROCK2+CAMK4+NFATC4+RYR3); and vii) without treatment (untreated) respectively.

FIG. 23 illustrates the tumour regression study demonstrating changes in relative tumour outgrowth volume (mm³) using a 4T1-induced breast tumour mouse model intravenously treated with i) no treatment (No treatment); ii) apatite-based matrix (CA); iii) free gemcitabine (Free Gemci); iv) apatite-based matrix with gemcitabine (CA Gemci); and v) pharmaceutical composition comprising gemcitabine (PEGylated CA Gemci) respectively.

FIG. 24 illustrates concentration of drug detected in 4T1-induced breast tumour mouse model, after intravenous delivery of drug via free gemcitabine (Free Gem), apatite-based matrix with gemcitabine (CA Gem) and pharmaceutical composition comprising gemcitabine (PEGylated Gem) respectively as in Example 4 (5).

FIG. 25 illustrates the result of measurement of concentration of accumulated drugs in liver, spleen, lung, brain, kidney and tumours of a 4T1 induced breast tumour mouse model after intravenous delivery of drug via free gemcitabine (Free Gem), apatite-based matrix with gemcitabine (CA Gem) and pharmaceutical composition comprising gemcitabine (PEGylated Gem) respectively as in Example 4 (5).

FIG. 26 illustrates the result of measurement of concentration of accumulated drugs in brain, heart, kidney, liver, lung, spleen, brain and tumour of a tumour mouse model after intravenous delivery of fluorescence siRNA-labelled apatite-based matrix at different amount of Fe²⁺/Fe³⁺ and Mg²⁺ ions as well as surface modifying agents.

FIG. 27 illustrates the result of measurement of average size of the apatite-based matrix with and without incorporation of citrate at concentration of 1 mM and 2 mM.

FIG. 28 illustrates the size and zeta potential measurement of pharmaceutical composition (drug loaded apatite-based matrix without surface modification). Drug-loaded apatite-based matrix (CA+drug) was used as control in (a) and (b) to be compared with the drug-loaded citrate-incorporated apatite-based matrix in (c) and (d).

FIG. 29 illustrates the drug binding efficiency (%) to apatite-based matrix (CA NP), citrate-incorporated apatite-based matrix (CMCA NP) and succinate-incorporated apatite-based matrix (SMCA NP).

### DETAILED DESCRIPTION OF THE INVENTION

While the present invention is described herein by way of example using embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claim. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes. Any discussion of documents, acts, materials, devices, articles and the like is included in the specification solely for the purpose of providing a context for the present invention. It is not suggested or represented that any or all of these matters form part of the prior art base or were common general knowledge in the field relevant to the present invention.

In this disclosure, whenever a composition or an element or a group of elements is preceded with the transitional phrase “comprising”, it is understood that we also contemplate the same composition, element or group of elements with transitional phrases “consisting of”, “consisting”, “selected from the group of consisting of, “including”, or “is” preceding the recitation of the composition, element or group of elements and vice versa.

The present invention is described hereinafter by various embodiments with reference to the accompanying drawing, wherein reference numerals used in the accompanying drawing correspond to the like elements throughout the description. This invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. These values and ranges are to be treated as examples only, and are not intended to limit the scope of the claims. In addition, a number of materials are identified as suitable for various facets of the implementations. These materials are to be treated as exemplary, and are not intended to limit the scope of the invention.

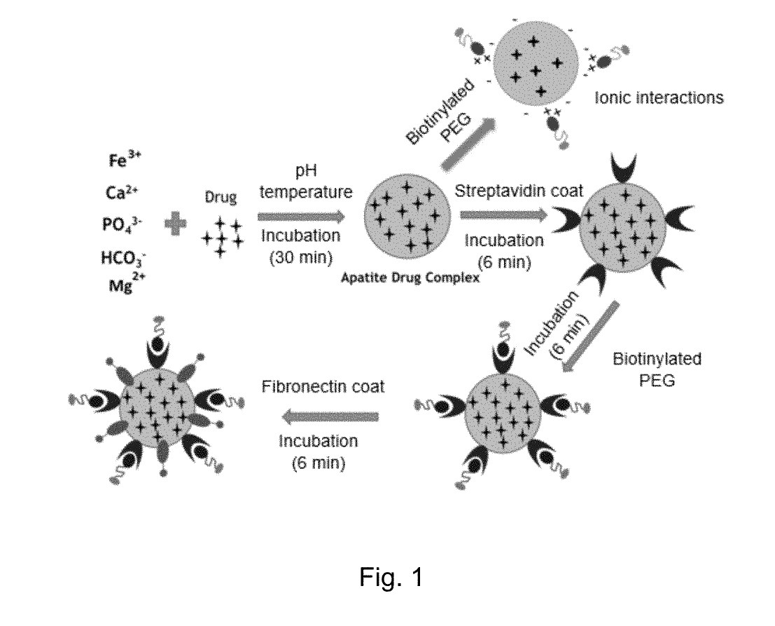

Referring to the drawings, the invention will now be described in more detail. FIG. 1 is a flow chart illustrating a formation process of a pharmaceutical composition in accordance with an embodiment of the present invention. The pharmaceutical composition comprises a pharmaceutically active substance, an apatite-based matrix, and a surface modifying agent.

In accordance with an embodiment of the present invention, the pharmaceutically active substance is selected from, but not limited to, the group consisting of drug, protein, nucleic acid and any combination thereof, for instance, a combination of drug and nucleic acid. The drug is, but not limited to, any potential therapeutic agent for a human disease, preferably an anti-tumour agent. The anti-tumour agent is selected from, but not limited to, the group comprising of antimetabolites, alkylating agents and antibiotics. The anti-tumour agent may also include enzymes, hormones, receptor antagonists or other similar substances. The antimetabolites are preferably, but not limited to, gemcitabine, methotrexate and fluorouracil, while the alkylating agents is preferably, but not limited to, cyclophosphamide. The antibiotics is preferably, but not limited to, doxorubicin. These anti-tumour agents can be used alone or in combination of two or more.

Further, in accordance with an embodiment of the present invention, the nucleic acid is, but not limited to, DNA, RNA, oligonucleotide or polynucleotide. The RNA is preferably, but not limited to, siRNA, miRNA, or antisense RNA. The siRNA is preferably, but not limited to, ESR1 siRNA, BCL-2 siRNA, ERBB2 siRNA, EGFR siRNA, ROS1 siRNA, SHC1 siRNA, ROCK2 siRNA, CAMK4 siRNA, RYR3 siRNA. Other gene silencing segment may also be used as the RNA. These nucleic acids may be used alone or in combination of two or more.

In accordance with an embodiment of the present invention, the apatite-based matrix comprises calcium ion, phosphate ion, hydrogen carbonate ion, magnesium ion and iron ion. The apatite-based matrix further comprises strontium ion, fluoride ion, barium ion or any combination thereof. In another embodiment of the present invention, the apatite-based matrix may further comprise a carboxyl group-containing molecule including citrate, succinate, pyruvate, lactate, alpha-ketoglutarate, oxaloacetate, fumarate and malate.

In accordance with an embodiment of the present invention, the surface modifying agent is, but not limited to, protein, polymer or combination thereof. The protein is, but not limited to, streptavidin, transferrin, fibronectin, collagen, albumin, lactoferrin, asialofetuin, lipoprotein or proteoglycan. The protein may further include any antibodies or fragments thereof. The polymer is, but not limited to, polyethylene glycol (PEG). Other PEG derivatives which have either positive or negative charges may also be used as the polymer. In a more preferred embodiment, one PEG chain is associated with a biotin moiety to form biotinylated PEG.

In accordance with an embodiment of the present invention, the size of the pharmaceutical composition is in a range of 5-1000 nanometer. The size of the pharmaceutical composition shall not be more than 1000 nm as the size is not suitable for administration purpose.

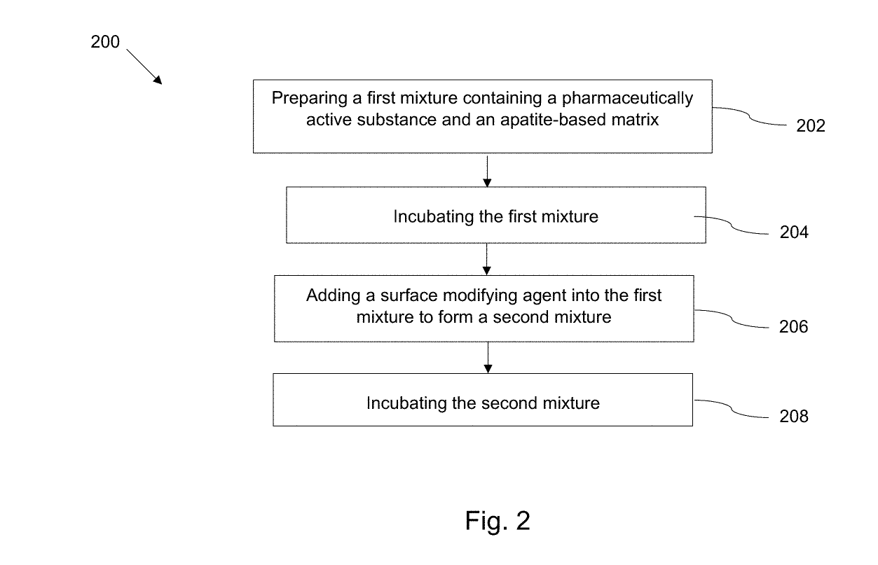

FIG. 2 illustrates a method of producing the pharmaceutical composition (200), in accordance with an embodiment of the present invention. The first step (202) comprises of preparing a first mixture containing a pharmaceutically active substance and an apatite-based matrix.

In accordance with an embodiment of the present invention, the apatite-based matrix in the first mixture comprises calcium ion, phosphate ion, hydrogen carbonate ion, magnesium ion and iron ion.

In accordance with an embodiment of the present invention, the apatite-based matrix further comprises strontium ion, fluoride ion, barium ion or any combination thereof.

In another embodiment of the present invention, the apatite-based matrix may further comprise a carboxyl group-containing molecule including citrate, succinate, pyruvate, lactate, alpha-ketoglutarate, oxaloacetate, fumarate and malate.

In accordance with an embodiment of the present invention, the first mixture has, but not limited to, a pH of 6.0-pH 8.0, preferably pH 7.5.

In accordance with an embodiment of the present invention, the apatite-based matrix is prepared by preparing a first solution that contains calcium ion, followed by adding the first solution into a second solution that contains phosphate ions, hydrogen carbonate ion, magnesium ion and iron ion. In other embodiment, the apatite-based matrix can be prepared by preparing a first solution that contains phosphate ion, followed by adding the first solution into a second solution that contains calcium ion, hydrogen carbonate ion, magnesium ion and iron ion.

In accordance with an embodiment of the present invention, the concentration of each ion is preferably, but not limited to, in a range of 0.1-100 millimolar.

In accordance with an embodiment of the present invention, the second solution further comprises sodium chloride and glucose. Further, the concentration of sodium chloride and glucose in the second solution is preferably, but not limited to, in a range of 10-1000 millimolar.

At step 204, the first mixture is subjected to a first incubation.

In accordance with an embodiment of the present invention, the first mixture is further added with at least one ion selected from strontium ion, fluoride ion and barium ion. In another embodiment of the present invention, the first mixture may be further added with at least one carboxyl group-containing molecule selected from citrate, succinate, pyruvate, lactate, alpha-ketoglutarate, oxaloacetate, fumarate and malate. In another further embodiment of the present invention, the first mixture may be added with at least one ion thereof and at least one carboxyl group-containing molecule thereof before the first incubation step (204).

In accordance with an embodiment of the present invention, the first mixture is further added with a protein-based surface modifying agent before the first incubation step (204). The protein-based surface modifying agent is preferably, but not limited to, fibronectin and collagen.

At step 206, the first mixture is further added with a surface modifying agent to form a second mixture.

At step 208, the second mixture is then subjected to a second incubation to form a pharmaceutical composition.

In accordance with an embodiment of the present invention, the surface modifying agent at step 206 is, but not limited to, protein, polymer or combination thereof. The protein is preferably, but not limited to, streptavidin, transferrin, fibronectin, collagen, albumin, lactoferrin, asialofetuin, lipoprotein or proteoglycan. Other antibodies and the fragments thereof may also be used as the protein. The polymer is preferably, but not limited to, polyethylene glycol (PEG). Other PEG derivatives which have either positive or negative charges may also be used as the polymer. In a preferred embodiment, one PEG chain is associated with a biotin moiety to form biotinylated PEG. The biotinylated PEG is further configured to be associated with streptavidin. In another embodiment, the biotinylated PEG may not be necessary to be associated with streptavidin.

In accordance with an embodiment of the present invention, each incubation is performed at a range of temperature of, but not limited to, 25° C. to 65° C., for, but not limited to, 6-30 minutes.

In accordance with an embodiment of the present invention, the pharmaceutical composition is dispersed in a pharmacologically acceptable solvent for use in treating human diseases, preferably tumour. The pharmacologically acceptable solvent is, but not limited to, a buffered cell culture medium solution or saline solution. The cell culture medium is, but not limited to, Dulbecco's Modified Eagle Medium (DMEM) or other cell culture medium.

In accordance with an embodiment of the present invention, the pharmaceutical composition is subjected to lyophilisation to obtain a powder form.

In accordance with an embodiment of the present invention, the pharmaceutical composition is subjected to high pressure condensation to obtain a solid dosage form. Hereinafter, examples of the present invention will be provided for more detailed explanation. It will be understood that the examples described below are not intended to limit the scope of the present invention.

### EXAMPLES

### Example 1

Production of Apatite-Based Matrix

(5) Preparation of Apatite-Based Matrix

Apatite-based matrix was formulated by adding an aqueous solution containing 6 mM calcium salt to a bicarbonate-buffered cell culture medium (Dulbecco's Modified Eagle Medium) containing 44 mM sodium bicarbonate, 0.9 mM inorganic phosphate, 2.5 mM ferric or ferrous salt, 0.8 mM magnesium salt, 110 mM sodium chloride (NaCl) and 25 mM glucose (pH 6 to 8). The mixture was then incubated at 37° C. for 30 minutes, resulting in formation of microscopically visible particles.

Generation of the apatite-based matrix in cell culture medium containing the above components along with amino acids and vitamins, indicates the possible adsorption of the amino acids and vitamins on the matrix surface.

(6) Characterization of Apatite-Based Matrix

The formation of apatite-based matrix was confirmed via chemical analysis, infrared spectroscopy, X-ray diffraction pattern (XRD) and X-ray fluorescence (XRF).

After generation of apatite-based matrix as described above, the apatite-based particles were then centrifuged and rinsed with distilled deionized water. This steps were repeated for 5 times before lyophilisation. Other apatite-based particles generated as described above, were also similarly lyophilized. Content of generated apatite-based particles was determined using liquid chromatography-mass spectrometry (LC-MS). Elemental analysis of the lyophilized particles proved that the apatite-based matrix was composed of 3% carbon, 17% phosphorus, 32% calcium, 0.026% magnesium, 0.01% iron.

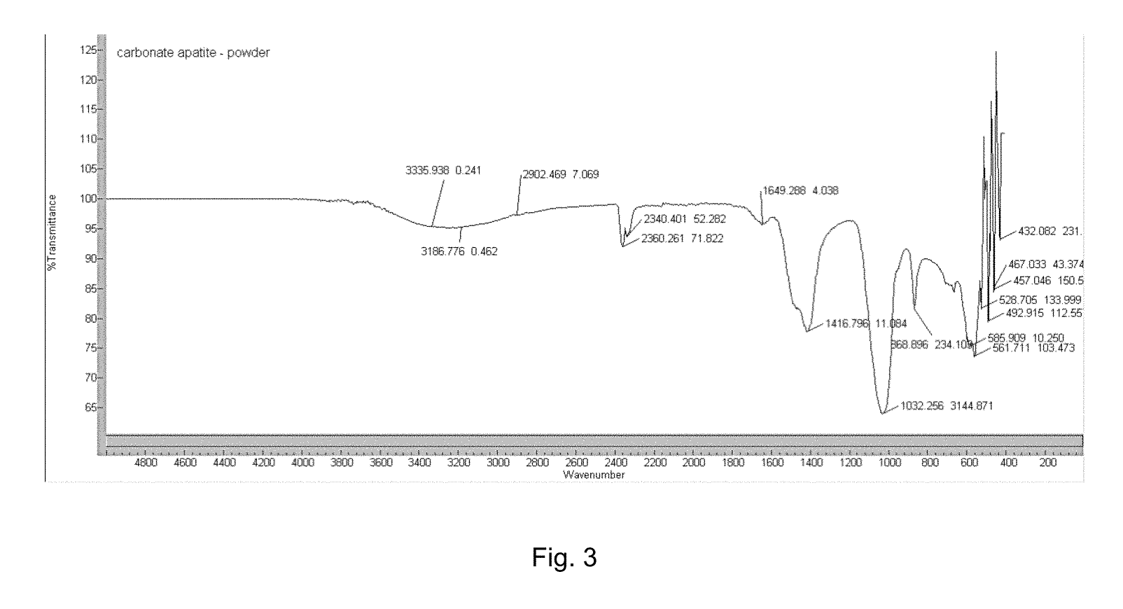

The formation of apatite-based matrix was further confirmed via Fourier Transform Infrared Spectroscopy (FT-IR) with the broad adsorption between 3343 and 3333 cm⁻¹ and 1657 and 1644 cm⁻¹, indicating adsorbed water as shown in FIG. 3. The spectrum is also showing peaks that represent carbonate at 1416 and 868 cm⁻¹, and phosphate at 1032, 585 and 561 cm⁻¹ in the poorly crystalline apatite, which is in agreement with the XRD result.

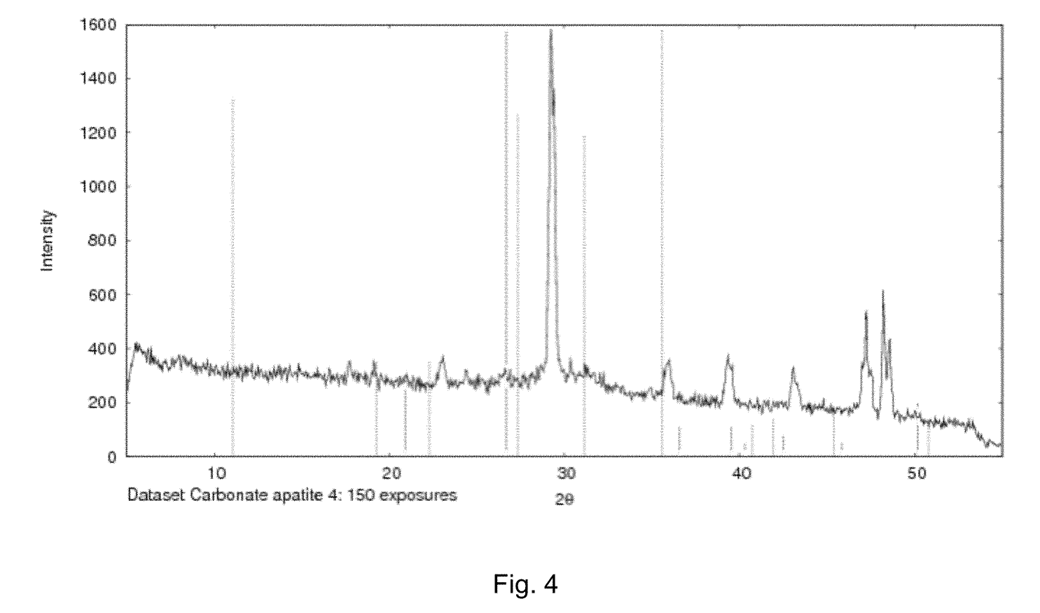

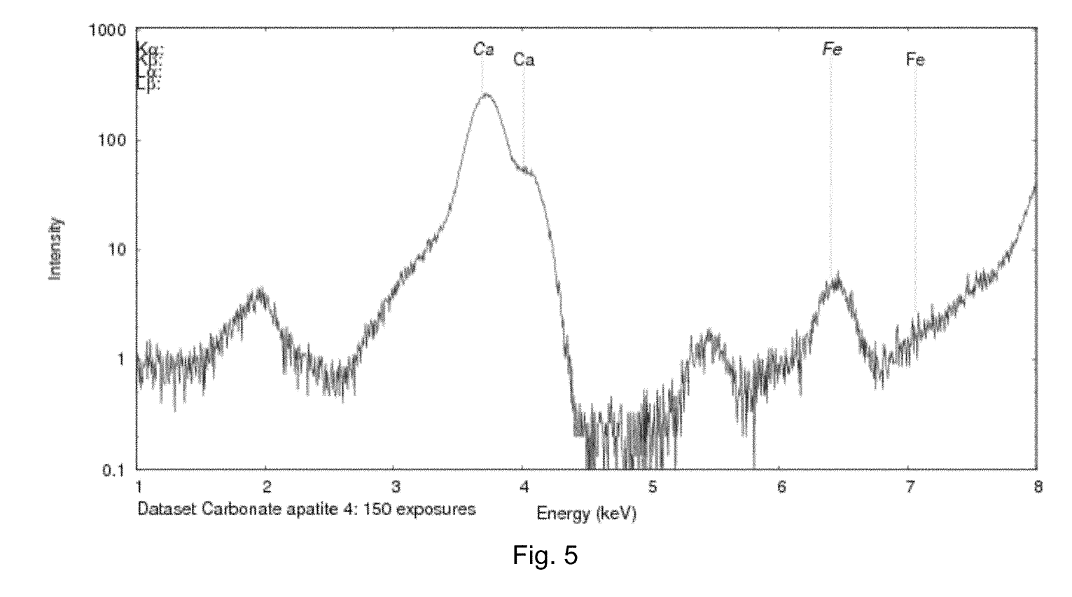

Result of XRD pattern also shows regular pattern of poorly crystalline apatite, which is represented by the broad diffraction peaks as shown in FIG. 4. XRF was run concurrently with the XRD in order to investigate the elements present in the powdered particle sample. As shown by FIG. 5, the presence of calcium and iron were successfully detected in the particle sample.

(7) Growth of Apatite-Based Matrix

Growth of apatite-based matrix was analysed via turbidity assessment and particle size measurement. Dissolution behaviour of the apatite particle in acid was further evaluated by pH adjustment using 1N hydrochloric acid (HCl) and turbidity assessment.

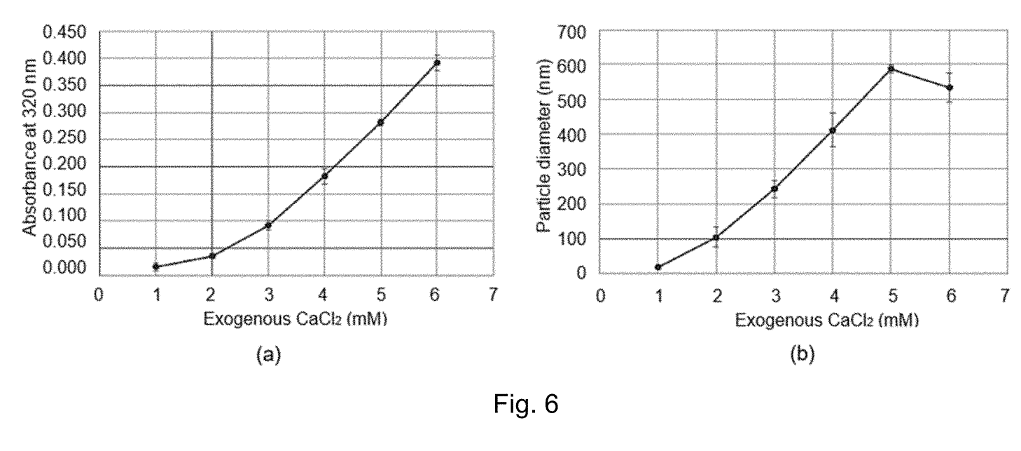

Apatite-based matrix was first prepared as described above and its growth was manipulated by adding increasing concentrations of exogenous calcium chloride from 1.0 to 6.0 mM to an aqueous solution containing 2.0 mM endogenous calcium chloride, 44 mM sodium bicarbonate, 2.5 mM ferric or ferrous salt, 0.9 mM inorganic phosphate, 0.8 mM magnesium salt, 110 mM sodium chloride (NaCl) and 25 mM glucose, followed by incubation at 37° C. for 30 minutes. Turbidity was measured by spectrophotometer using absorbance at 320 nm and the particle diameter was measured using Zeta sizer machine in nm units. The result is shown in FIGS. 6 (a) and (b).

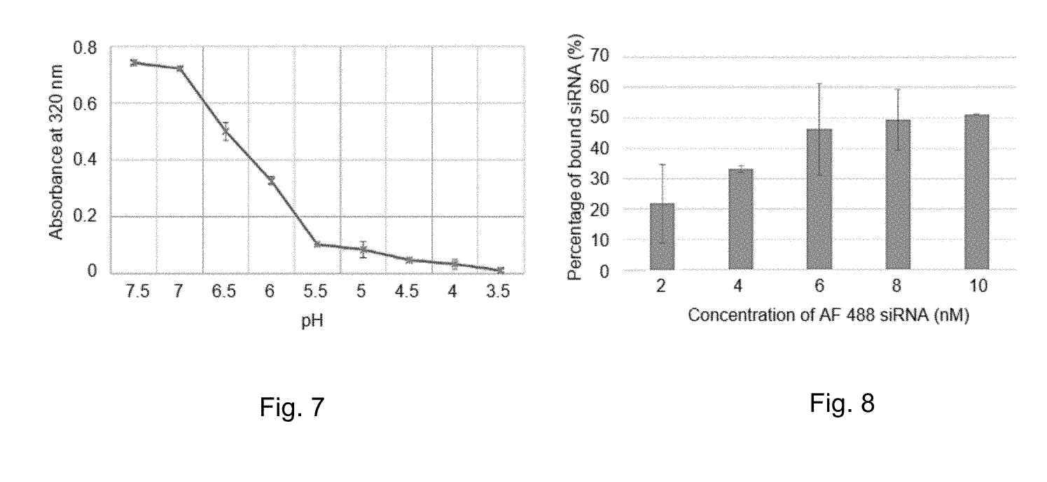

The acid dissolution test was then performed by pH adjustment from pH 7.5 to pH 3.5 in which the amount of 1N HCl was increased gradually, and the measurement of turbidity was taken using absorbance at 320 nm for each specific pH. The result is indicated in FIG. 7.

The results in FIGS. 6 (a) and (b) show that the apatite-based matrix growth could be controlled by changing one or more of the active components, such as calcium chloride, sodium bicarbonate, ferric or ferrous salt, inorganic phosphate and magnesium salt, thereby providing more driving force for the reaction of apatite-based matrix formation to proceed. Thus, an increasing trend was observed both for the turbidity and the particle size as the concentration of exogenous calcium chloride increases while concentrations of other components remain the same.

While particle size is crucial for favourable pharmacokinetics, enabling the apatite-based matrix to overcome their opsonisation by macrophages, release of the drugs bound to the apatite-based matrix is also of utmost importance after internalization of the particles by target cells via endocytosis. As shown in FIG. 7, the apatite-based matrix could be dissolved at acidic pH of the endosomes, suggesting that the apatite-based matrix would be able to facilitate drug release through self-dissolution in the acidic compartments.

(8) Binding Affinity of Apatite-Based Matrix

IV. Nucleic-Acid Based Drug

Apatite-based matrix as prepared above using 7 mM calcium chloride (CaCl₂) was allowed to interact with AllStars Negative siRNA AF 488 to form complexes. The complexes were then centrifuged at 13,000 rpm for 15 min and supernatant was discarded without disturbing the pellet. 100 μl of media was added to pellet to form a suspension and the suspension were collected and transferred into a 96-well plate of black OptiPlate. The plate was taken to a fluorescence microplate reader to measure the fluorescence signal in order to determine the percentage of bound siRNA (%) onto apatite-based matrix for each siRNA concentration as the concentration of the siRNA AF 488 increased from 2 to 10 nM. The percentage of bound siRNA onto apatite-based matrix demonstrated changes in binding affinity of apatite-based matrix towards siRNA.

As shown in FIG. 8, the binding affinity of apatite-based matrix towards siRNAs increased as the concentration of fluorescence siRNA (AF 488 siRNA) increased. The negatively-charged phosphate backbone of the siRNA might have interacted with the positively-charged (Ca/Mg/iron-rich) domains of the apatite-based matrix.

V. Small Molecule Drug

Apatite-based matrix as prepared above was allowed to interact with three types of drugs including cyclophosphamide (Cyp), methotrexate (Mtx) and 5-fluorouracil (5-FU) to form complexes. The complexes were then centrifuged at 13,000 rpm for 15 min and supernatant was discarded without disturbing the pellet. 100 μL of media was added to pellet to form a suspension and the suspension was used to perform high performance liquid chromatography (HPLC) analysis to estimate the concentration of drugs that could be adsorbed to the apatite-based matrix and also to evaluate the interaction efficiency of drugs with apatite-based matrix. The concentration of the drugs present in the supernatant was calculated from the peak area, using the standard curves. Data were represented as interaction efficiency (%) of drugs with apatite-based matrix, calculated using the following formula:

$$\% Interaction efficiency = \frac{{[ X ]}_{free drug} - {[ X ]}_{CA - drug}}{{[ X \}}_{initial}} \times 100$$

Where [X]_{free drug} and [X]_{CA-drug} are the concentrations of free drug and drug-loaded apatite-based matrix in the supernatant calculated form the standard curves and [X]ᵢₙᵢₜᵢₐₗ is the total concentration of drugs used to perform HPLC or the total concentration initially mixed for preparation of apatite-drug formulations. Each experiment was done in triplicate and shown as mean±SD.

The result is tabulated and shown in Table 1.

TABLE 1
Interaction efficiency (%) of drugs with the apatite-based matrix
Interaction efficiency (%) of each drug with apatite-based matrix

| Mtx | Cyp | 5-FU |
|---|---|---|
| 1.73 ± 0.85 | 13.10 ± 5.47 | 0 |

According to Table 1, there were variations in binding affinity depending on the drugs used, with cyclophosphamide (Cyp) showing higher affinity than methotrexate (Mtx), while 5-fluorouracil (5-FU) did not show any affinity.

Size of drug-apatite complex was further measured to observe the changes in the size of apatite-based matrix loaded with doxorubicin and cyclophosphamide respectively.

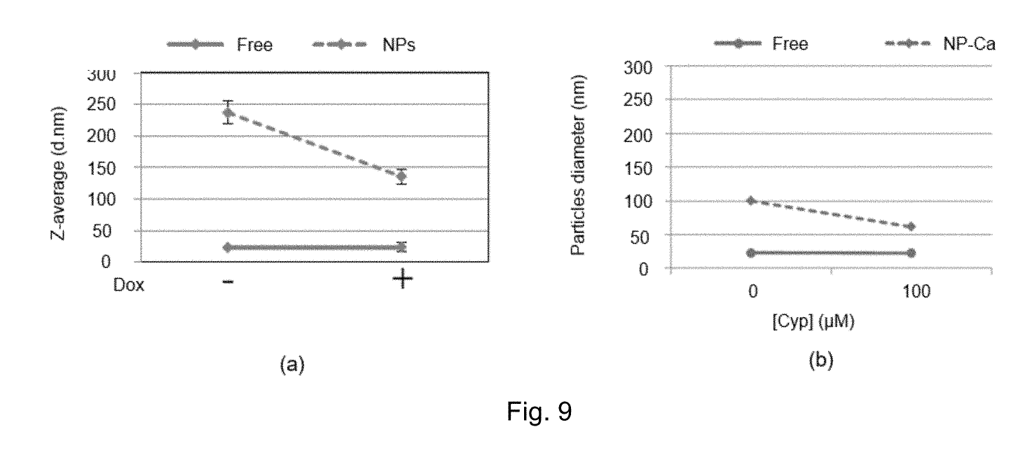

The result as indicated in FIG. 9 (a) shows that the average diameter of the apatite-based matrix was reduced from approximately 236.95 nm to 135.55 nm upon complexing with water soluble drug, doxorubicin, and the similar pattern can be observed in cyclophosphamide as well in FIG. 9 (b). Free media with or without doxorubicin and devoid of any apatite-based particle did not show any difference in the particle size.

VI. Combination of Drugs

Apatite-based matrix as prepared above were allowed to interact with a combination of drugs including doxorubicin and siRNA to form complexes. The following procedure was the same as described above to measure the fluorescence signal in order to determine the percentage of bound siRNA (%) onto the apatite-based matrix loaded with doxorubicin. The percentage of bound siRNA onto apatite-based matrix demonstrated changes in binding affinity of apatite-based matrix towards siRNA.

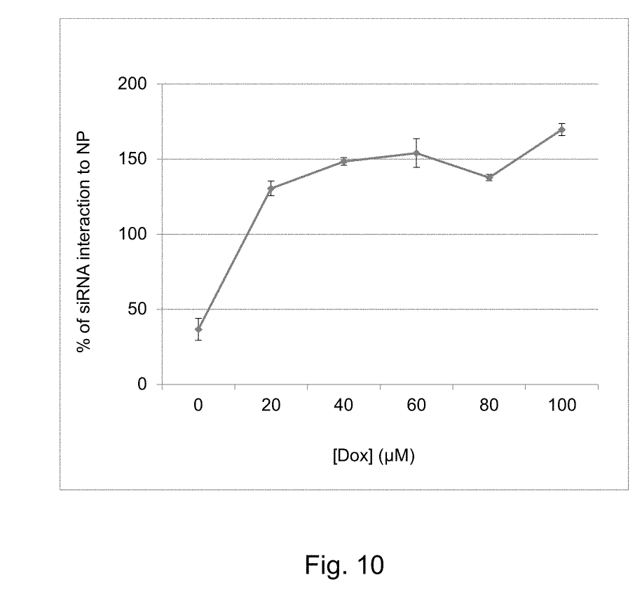

As shown in FIG. 10, co-delivery of drugs and siRNA using apatite-based matrix of the present invention is highly prospective as more siRNA interactions with apatite-based matrix could be facilitated by the presence of drugs, as indicated by an increase in the binding affinity of apatite-based matrix for the siRNA in the presence of doxorubicin. However, the interactions did not change to a significant extent with different concentrations of doxorubicin.

### Example 2

### Ions Substitution in Production of Apatite-Based Matrix

(6) Fe²⁺/Fe³⁺ and Mg²⁺

Role of Fe³⁺ and Mg²⁺ in production of apatite-based matrix was tested by increasing the amount of each of the two salts in the apatite-based matrix preparation while reducing the total amount of Ca²⁺.

Apatite-based matrix formulated with 44 mM sodium bicarbonate, 2 mM calcium salt, 2.65 mM ferric salt, 0.9 mM inorganic phosphate, 1.8 mM magnesium salt, 110 mM sodium chloride (NaCl) and 25 mM glucose were allowed to interact with luciferase plasmid. Then, MCF-7 and 4T1 cells were transfected with the luciferase plasmid-carrying apatite-based matrix, followed by observation and measurement on fluorescence (luciferase expression level) after transfection of MCF-7 and 4T1 cells. The luciferase expression level was measured in relative light units (RLU) per mg of protein. The amount of each salt (Fe²⁺/Fe³⁺ and Mg²⁺) was manipulated while reducing total amount of Ca²⁺.

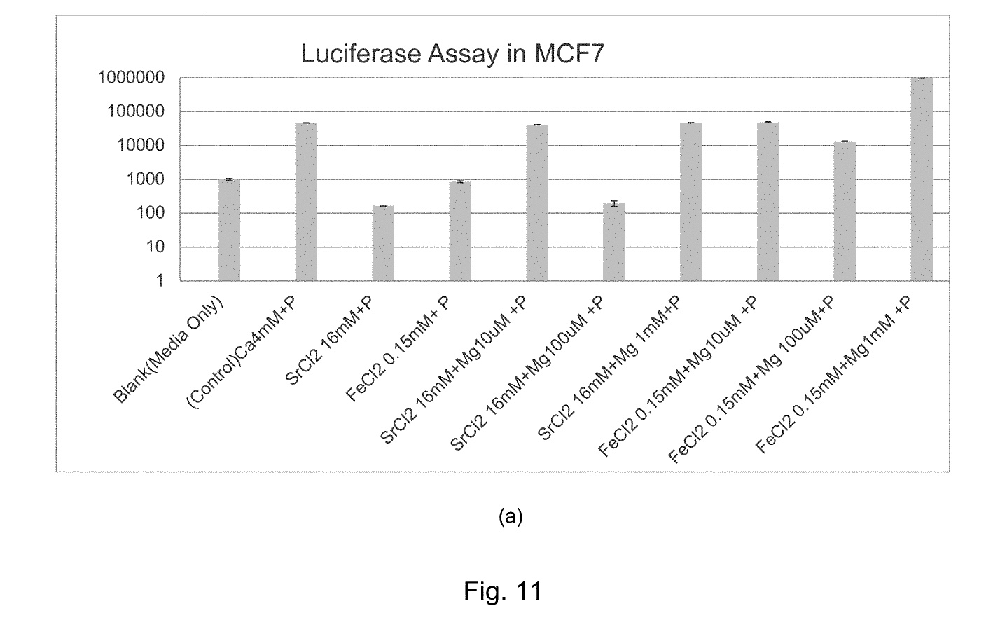

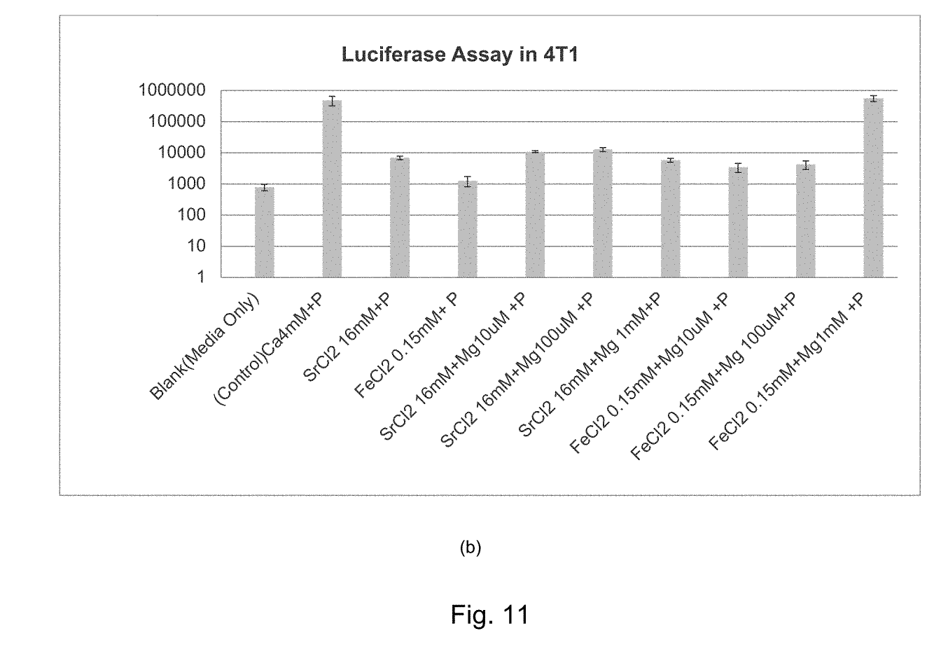

As shown in FIGS. 11(a) and (b), inclusion of additional Fe²⁺/Fe³⁺ ion in the apatite-based matrix formation reduced the transfection efficiency. However, by inclusion of both Fe²⁺/Fe³⁺ and Mg²⁺ ion, gene expression was shown to be dramatically accelerated compared to the control. This could be possibly because inclusion of Mg²⁺ ion may significantly reduce the particle diameter while the Fe²⁺/Fe³⁺ ion may increase the particle diameter simultaneously.

(7) Ca²⁺ and Sr²⁺ Effects of Ca²⁺ and Sr²⁺ on apatite-based matrix formation were analysed by adding exogenous CaCl₂ and SrCl₂ in apatite-based matrix fabrication respectively, while keeping other salts being constant. The size of the apatite-based matrix formed with exogenous CaCl₂ and SrCl₂ were measured, followed by evaluation on the interaction efficiency (%) of apatite-based matrix with drugs which is indicated in Table 2.

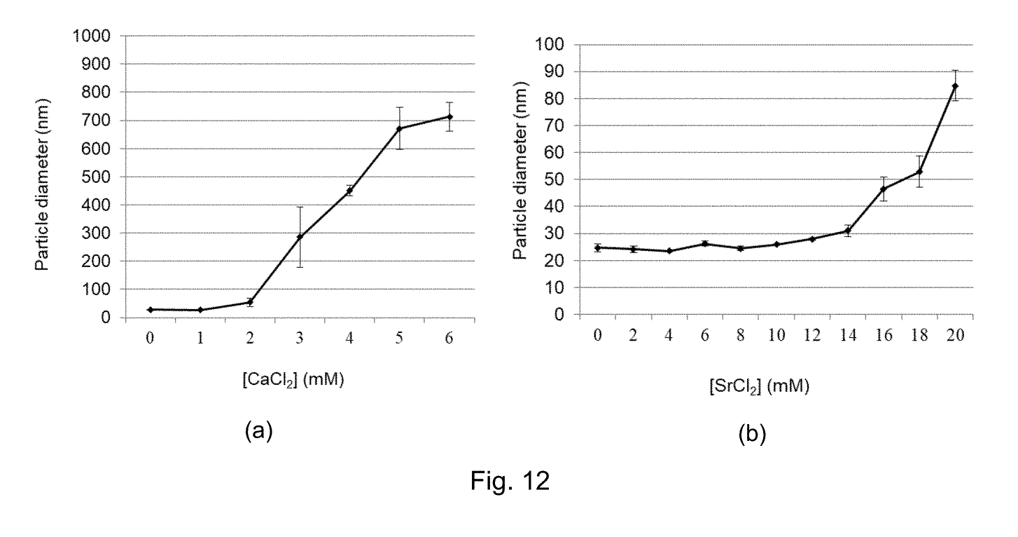

As shown in FIG. 12 (a), calcium has a tendency to flocculate at higher concentrations and forms larger particles probably by reducing electrostatic repulsion and thus enabling the particles to come into close proximity and form aggregation. Thus, Ca²⁺ may increase the particle diameter with their concentrations in the reaction mixture. In contrast, the smaller size of the particles formed by Sr²⁺ might be attributed by incorporation of more carbonate ion in the loosen lattice network formed by strontium, as indicated in FIG. 12 (b).

Higher affinity of methotrexate and 5-fluorouracil towards the apatite-based matrix formed with exogenous SrCl₂ than the apatite-based matrix formed with exogenous CaCl₂ as shown by HPLC results, is also reflected by the significant changes in apatite-based matrix growth kinetics as a result of the possible apatite-drug interactions (Table 2). On the contrary, cyclophosphamide was more likely to bind with the apatite-based matrix formed with CaCl₂ than those with SrCl₂ as revealed by HPLC analysis, which was not very evident from the result shown in Table 2.

TABLE 2
Interaction efficiency (%) of each drug with the apatite-based matrix
formed with CaCl₂ and SrCl₂.

| | Mtx | Cyp | 5-FU |
|---|---|---|---|
| CaCl₂ | 1.73 ± 0.85 | 13.10 ± 5.47 | 0 |
| SrCl₂ | 27.76 ± 5.62 | 11.19 ± 8.16 | 1.21 ± 0.56 |

Further, the treatment effects of apatite-cyclophosphamide complex on 4T1 induced mouse model of tumour were evaluated by using apatite-based matrix formed with exogenous CaCl₂.

4T1 cells were first inoculated subcutaneously on the mammary pad of mice. Mice were treated intravenously through tail-vein injection by administering 100 μL of each solution as follows: i) untreated aqueous solution; ii) solution containing apatite-based matrix formed in 90 mM of exogenous CaCl₂ with other salt concentrations being constant; iii) solution containing 0.17 mg/Kg free cyclophosphamide; and iv) solution containing apatite-cyclophosphamide complex formed with 90 mM CaCl₂ with other salt concentrations being constant, respectively. As the tumour volume reached to 13.20±2.51 mm³, second administration was given after 3 days from first administration. The body weight and tumour outgrowth volume were monitored accordingly.

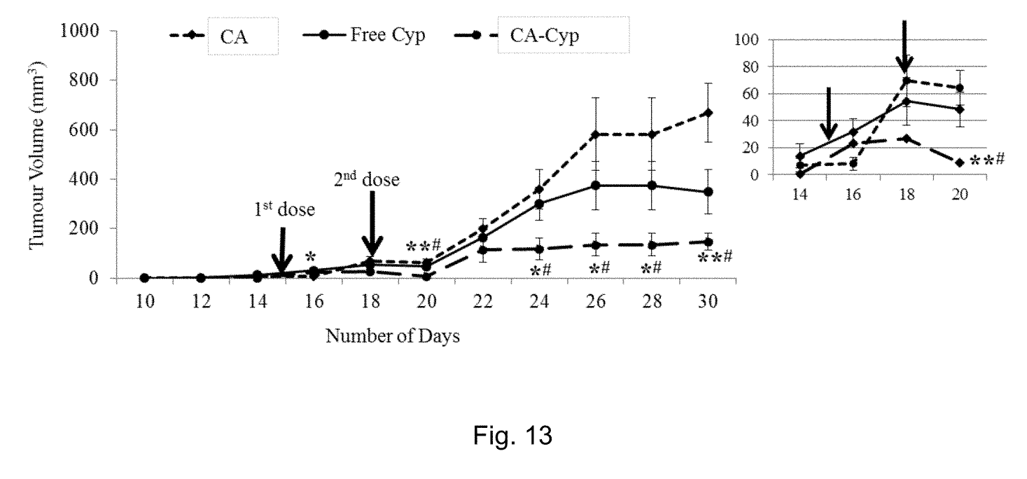

The result is indicated in FIG. 13. Six mice were used per group and data were represented as mean±SD. Values were significant when p<0.05 (*) and p<0.01 (**) compared to apatite-based matrix treated group; p<0.05 (#) when compared to free cyclophosphamide group.

As shown in FIG. 13, the large size particles (˜600 nm) which are more efficiently accumulated in liver, have significantly reduced the tumour volume when compared to the small particles (˜200 nm), after intravenous injection into 4T1 induced murine breast cancer model at a very low dose (0.17 mg/Kg) of the apatite-cyclophosphamide complex.

### Example 3

### Surface Modification in Pharmaceutical Composition Production

(5) Bio-Distribution of Pharmaceutical Composition

Bio-distribution of pharmaceutical composition comprising apatite-based matrix incorporated with AllStars Negative AF 488 siRNA with involvement of fibronectin and transferrin coating on various organs was examined by using the procedure below.

4T1 tumour-induced BALB/c mice were treated intravenously through tail-vein injection by administering 100 μL of pharmaceutical composition comprising surface-coated apatite-based matrix formed with incorporation of 1 μM siRNA when the tumour volume reached approximately 13.20±2.51 mm³. Mice were sacrificed for 1, 2 or 4 hours post treatment, followed by organs harvesting and lysis. Tissue lysates were centrifuged at 15,000 rpm for 30 minutes at 4° C. and 100 μL supernatants were taken for observation of fluorescence activity available in each organs by measuring fluorescence activity per 500 mg of tissue mass.

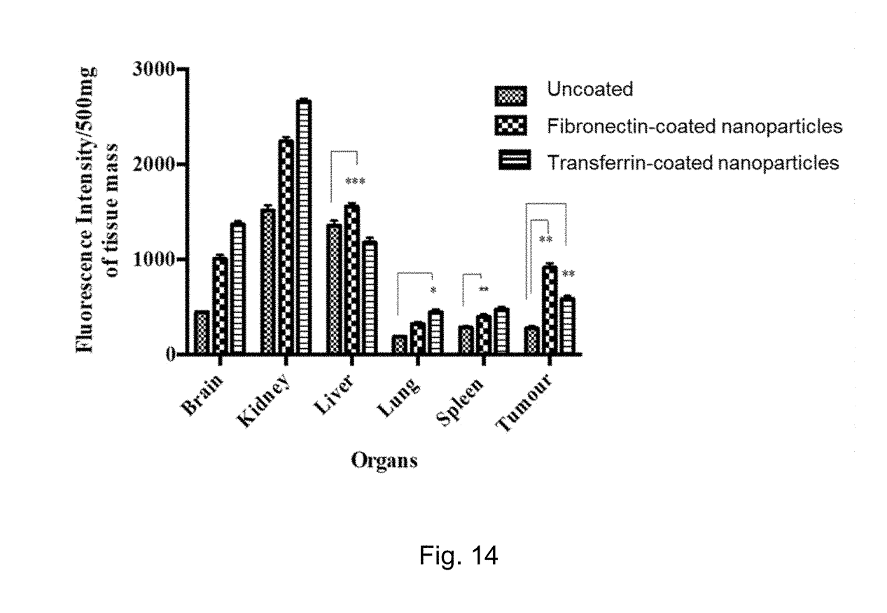

The result is shown in FIG. 14. Five mice per group were randomly assigned after tumour induction, and data was represented as mean±SD of the fluorescence intensity per 500 mg of tissue mass. Significance value was represented by p<0.0001 (****), p<0.001 (***), p<0.01 (**) and p<0.05 (*) as compared to uncoated apatite-based matrix for each respective organs.

(6) Interaction Between Surface Modifying Agent and Apatite-Based Matrix

Evaluation on interaction of surface modifying agent including biotinylated PEG, streptavidin and fibronectin with apatite-based matrix was carried out by detecting their presence via SDS-PAGE and silver staining procedure.

An apatite-based matrix surface-modified with biotinylated PEG, streptavidin and fibronectin was prepared as described previously, followed by centrifugation performed at 4° C. with 13,000 rpm for 15 min. The supernatant was discarded and the pellet was resuspended in 100 uL DMEM solution. Then, 6 uL of samples and loading dye with 1:1 ratio were loaded into each gel well (BioRad Precast Gels 7.5%) and run through SDS PAGE at 60 V for 1 hour. The resulting gel was processed through silver staining. Each control was included (streptavidin, biotinylated PEG, fibronectin and apatite-based matrix).

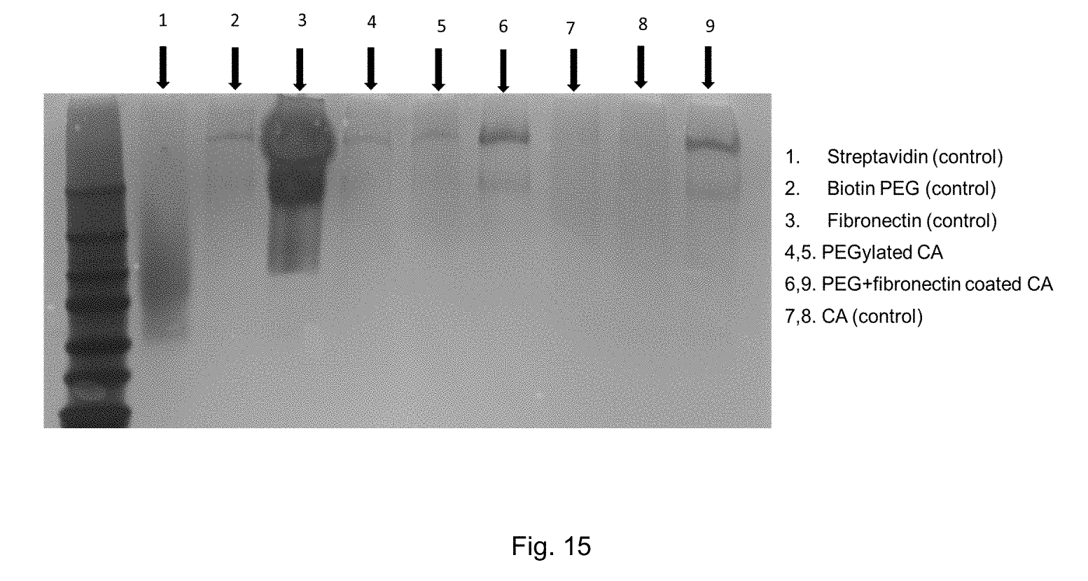

As shown in FIG. 15, both biotinylated PEG and fibronectin were proven to be interacted with the apatite-based matrix, although the signal for streptavidin was not at the detectable level. The direct binding of biotinylated PEG to the surface of apatite-based matrix via electrostatic interactions could not be ruled out, since biotin moiety possesses protanable amine groups and ionisable carboxyl group.

A further investigation on surface modification on the apatite-based matrix including combination of streptavidin and biotinylated PEG (streptavidin-biotinylated PEG) and biotinylated PEG alone were performed by assessing their potential ability of preventing serum protein binding to the apatite-based matrix in the pharmaceutical composition.

First, both surface modified and unmodified apatite-based matrix prepared as described above were treated with 20% Fetal Bovine Serum (Gibco), followed by incubation for 30 mins at 37° C. The apatite-based matrix were surface-modified with streptavidin-biotinylated PEG and biotinylated PEG alone respectively. Following centrifugation at 13,000 rpm for 15 mins, the supernatant was removed and rinsed with double distilled water. The pellet was dissolved with 100 ul of 50 mM EDTA in water. 6 ul of samples and loading dye with 1:1 ratio were loaded into each gel well (BioRad Precast Gels 7.5%) and run through SDS-PAGE at 60 V for 1 hour. The resulting gel was further processed for staining using BlueBANDit protein stain (AMRESCO). Fetal Bovine Serum and apatite-based matrix were used as control respectively.

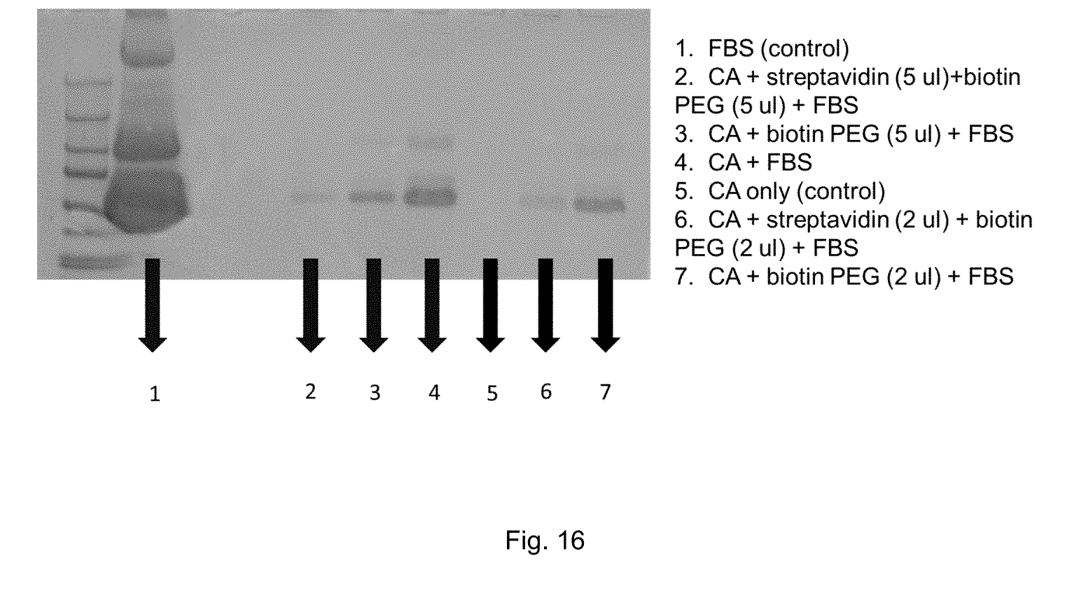

As shown in FIG. 16, streptavidin-biotinylated PEG could more significantly prevent serum protein binding to the apatite-based matrix than biotinylated PEG alone, indicating that biotinylated PEG could also directly bind to the apatite-based matrix in the pharmaceutical composition without the aid of streptavidin.

(7) Influence of Surface Modification on Size and Surface Charge of Pharmaceutical

Composition

Influence of surface modification on size and surface charge of pharmaceutical composition was evaluated using size and zeta potential measurement of pharmaceutical composition (surface-modified, drug loaded apatite-based matrix). Surface-modified apatite-based matrix without drug was also included in the evaluation. The surface modifying agent(s) used including streptavidin (+strep), streptavidin and biotinylated-PEG (+strep+PEG), and a combination of streptavidin, biotinylated-PEG and fibronectin (+strep+PEG+fib). Apatite-based matrix alone and drug-loaded unmodified apatite-based matrix were included as control. Inclusion of streptavidin and biotinylated PEG onto apatite-based matrix is denoted as PEGylation.

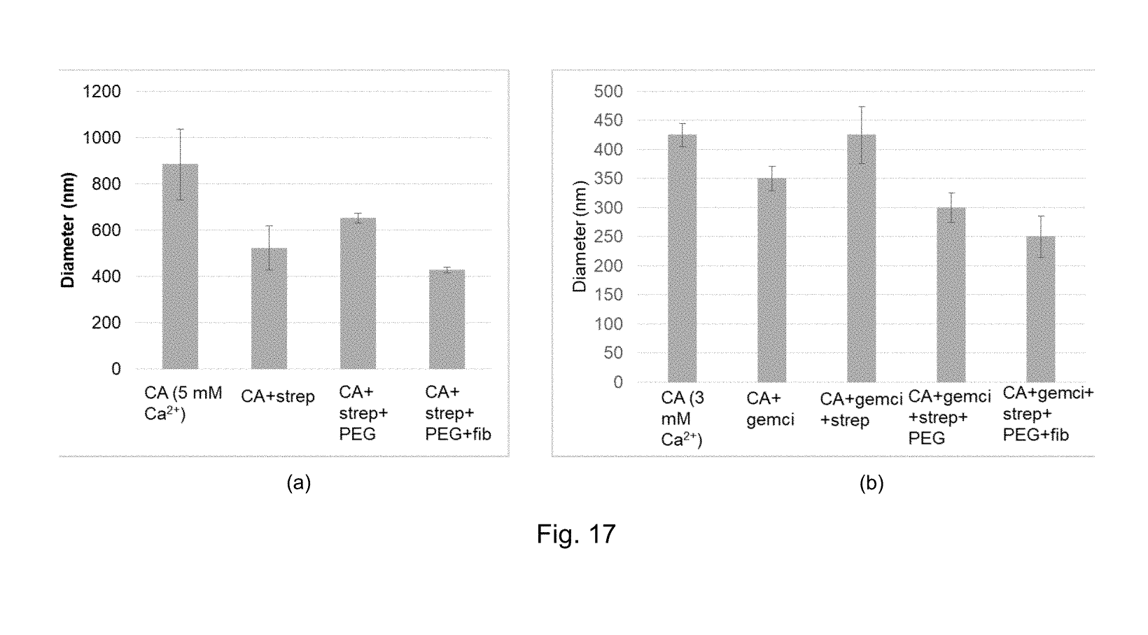

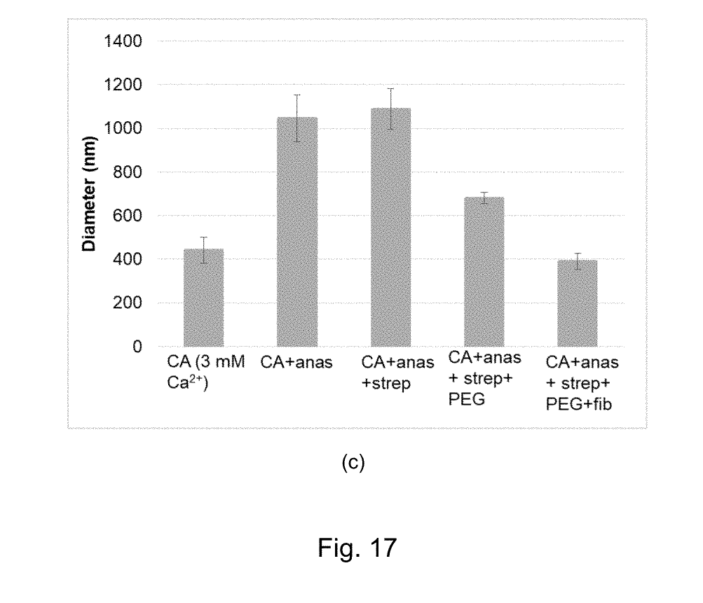

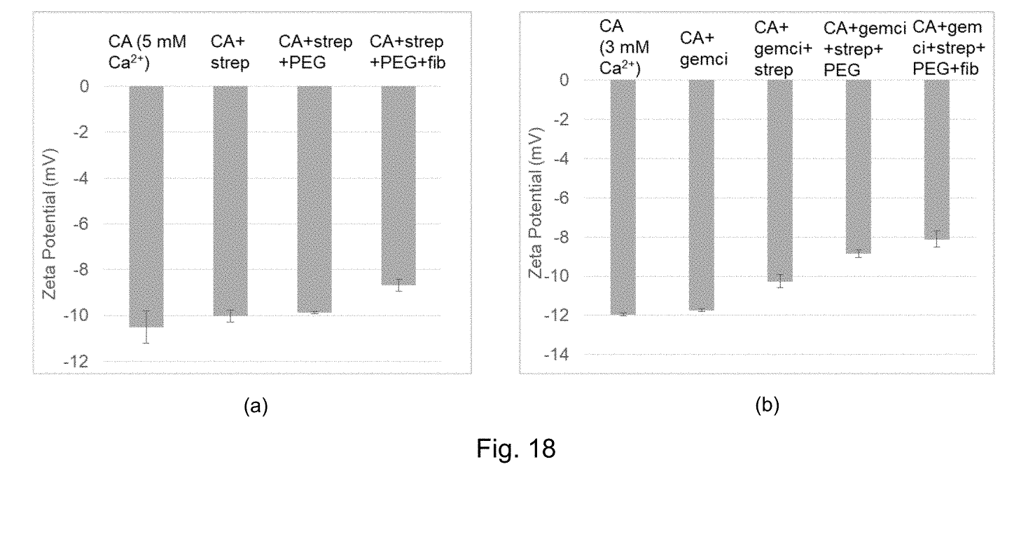

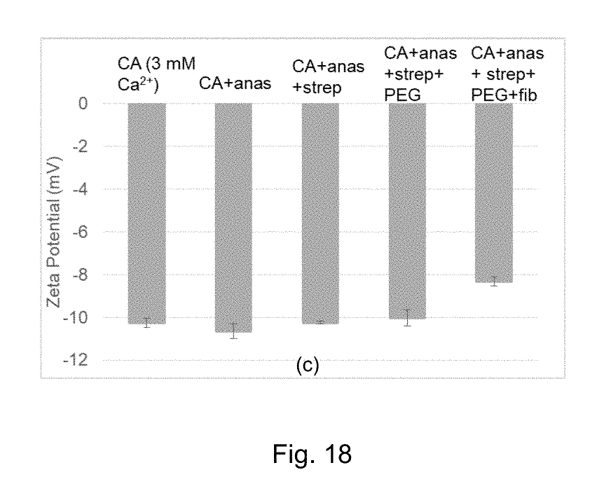

While the initial average diameter of the unloaded apatite-based matrix prepared was 820 nm, the surface-modified apatite-based matrix showed a decreasing pattern in its size when it reached ±610.5 nm after PEGylation, and ±402 nm after PEGylation and addition of fibronectin coating respectively (FIG. 17a). Fibronectin, a cell specific ligand was attached to the apatite-based matrix in order to facilitate receptor-mediated endocytosis based on fibronectin-integrin interaction. On the other hand, the zeta potential of the surface-modified apatite-based matrix shows little changes compared to the apatite-based matrix alone, turning out to be slightly more electropositive after the modification (FIG. 18a).

For gemcitabine-loaded apatite-based matrix (CA+gemci), PEGylation demonstrated slight effect on size reduction from initial average diameter approximately ±340 nm at 1 uM drug concentration to ±285.4 nm (FIG. 17b). Further effects on size reduction reaching ±220 nm was observed after addition of fibronectin coating. Zeta potential also slightly increased from −12 mV to −8 mV after PEGylation and addition of fibronectin coating (FIG. 18b).

PEGylation appeared to give a significant size reduction for anastrozole-loaded apatite-based matrix (CA+anas) from initial average diameter approximately ±1000 nm at 1 uM drug concentration to ±621.4 nm, whereas addition of fibronectin coating further decreased the size to ±410 nm (FIG. 17c). The zeta potential slightly increased from −10 mV to −8 mV with the aid of PEGylation and fibronectin coating (FIG. 18c).

(8) Cellular Uptake of Pharmaceutical Composition with Surface Modification

HPLC was performed to determine time-dependent cellular uptake (%) of pharmaceutical composition and also drug-loaded unmodified apatite-based matrix in MCF7 cell line. Apatite-based matrix was formulated with different Ca²⁺ concentrations (7 mM, 8 mM and 9 mM) and 20 uM of drug. Free drug (20 uM) was used as control. The results are indicated in Table 3 and 4.

Referring to Table 3 and 4, there was an apparent increase in the cellular uptake of the pharmaceutical composition (coated CA) at time intervals of 1, 4 and 24 hours when compared to the free drug and the drug delivered by unmodified apatite-based matrix, indicating that cell specific targeting and PEGylation facilitated more internalization of pharmaceutical composition apatite-based matrix by the cells. Longer treatment time also increased the cellular uptake until reaching almost 100% after 24 hours. Since large particles were less effectively endocytosed than small particles, the tendency of gemcitabine in reducing the size of pharmaceutical composition after incorporation could be a factor that enables higher cellular uptake, as shown by both unmodified and surface-modified apatite-based matrix that facilitated more drug uptake at 1 hr time point compared to free drugs (Table 3). At 4 hr time point, the pharmaceutical composition showed higher cellular uptake than the unmodified apatite-based matrix and the free drug, which could be due to the influence of surface modifying agent in regulating the size of pharmaceutical composition and also in facilitating its specific binding to the cell membrane as shown in FIG. 17 and FIG. 18. On the other hand, cellular uptakes of anastrozole delivered by the unmodified apatite-based matrix and the pharmaceutical composition were found to be more significantly improved compared to that of the free drug at time interval of 1 hr and 4 hr, shedding light on the fact that anastrozole has no role in inhibiting growth of apatite-based matrix which hampers the cellular uptake (Table 4). Interestingly, the pharmaceutical composition played a more powerful role in accelerating the drug uptake compared to the unmodified apatite-based matrix at those two time points, which could be due to the influences of reduced size of apatite-based matrix as a result of surface modification in agreement with the earlier finding (shown in FIG. 17c). It also showed the importance of the pharmaceutical composition comprising fibronectin in accelerating integrin-mediated specific cellular uptake.

TABLE 3
Time-dependent cellular uptake (%) of pharmaceutical composition comprising
gemcitabine and also gemcitabine-loaded unmodified apatite-based matrix in MCF7 cell line.

| | | CA | Coated | | Coated | CA | Coated |
|---|---|---|---|---|---|---|---|
| 20 | Free | (Ca²⁺ | CA | CA | CA | (Ca²⁺ | CA |
| uM | gemcitabine | 7 mM) | (Ca²⁺ 7 mM) | (Ca²⁺ 8 mM) | (Ca²⁺ 8 mM) | 9 mM) | (Ca²⁺ 9 mM) |
| 1 h | 30.5% ± 1.2 | 52% ± 2 | 57% ± 1 | 55.5% ± 3.52 | 57.1% ± 1.06 | 60% ± 2 | 61.2% ± 1.3 |
| 4 h | 50.75% ± 2.3 | 79% ± 3.42 | 86.65% ± 2 | 80.8% ± 2.48 | 83.66% ± 1.6 | 81.2% ± 4.5 | 84% ± 2.4 |
| 24 h | 100% | 100% | 100% | 100% | 100% | 96.4% ± 2.5 | 95.4% ± 3.2 |

TABLE 4
Time-dependent cellular uptake (%) of pharmaceutical composition comprising
anastrozole and also anastrozole-loaded unmodified apatite-based matrix in MCF7 cell line.

| | | | Coated | CA | Coated | | Coated |
|---|---|---|---|---|---|---|---|
| 20 | Free | CA | CA | (Ca²⁺ | CA | CA | CA |
| uM | anastrozole | (Ca²⁺ 7 mM) | (Ca²⁺ 7 mM) | 8 mM) | (Ca²⁺ 8 mM) | (Ca²⁺ 9 mM) | (Ca²⁺ 9 mM) |
| 1 h | 18.5% ± 2.4 | 30% ± 2.4 | 51% ± 2 | 16.5% ± 1.4 | 28.24% ± 2.7 | 11.3% ± 1.82 | 33.4% ± 2.76 |
| 4 h | 47.75% ± 2.3 | 51% ± 1.7 | 84.30% ± 1.6 | 43.2% ± 2.43 | 56.35% ± 1.8 | 31.5% ± 2.6 | 48.2% ± 1.6 |
| 24 h | 89.5% ± 3 | 93% ± 2 | 100% | 75.4% ± 2.6 | 81% ± 1.6 | 71.42% ± 1.8 | 73.45% ± 2.2 |

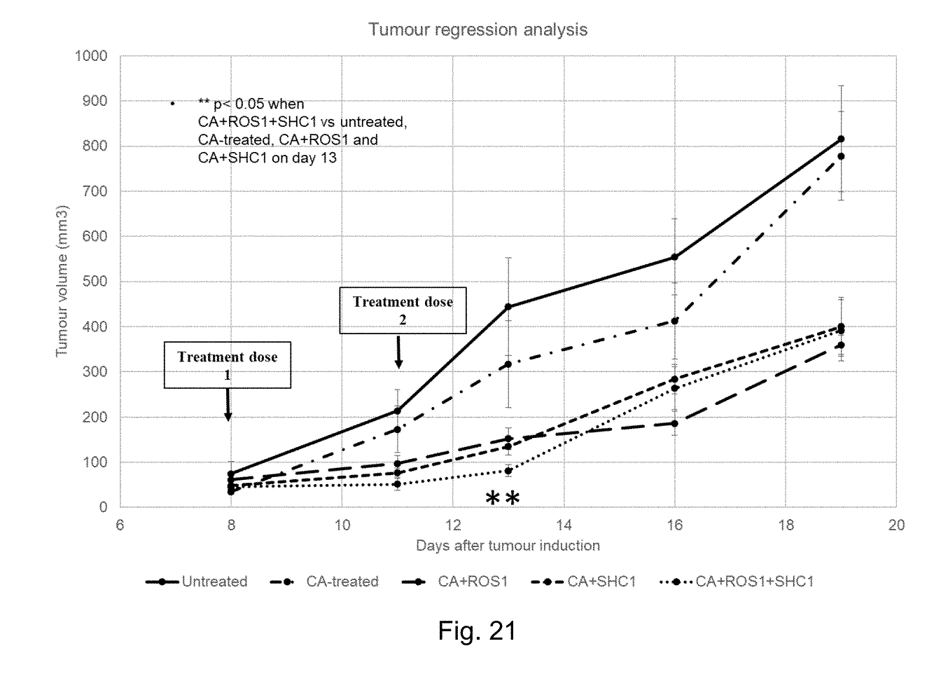

Further, in vitro chemosensitivity assay and in vivo tumour regression study also showed that pharmaceutical composition presented higher cytotoxicity (Table 5-8) and tumour regression effects (FIG. 21) than that of unmodified apatite-drug complexes and free drug, indicating that surface modification successfully created optimum particles size with the consequence of more effective uptake along with favourable pharmacokinetics of the pharmaceutical composition.

TABLE 5
Enhancement of cytotoxicity (%) for gemcitabine-loaded unmodified
apatite-based matrix (A) and pharmaceutical composition comprising
gemcitabine (B) in MCF 7 cell line, in an increasing concentration.

| | 100 pM | 1 nm | 10 nM | 100 nM | 1 uM |
|---|---|---|---|---|---|
| A | 1.4 ± 1.8 | 2.8 ± 2.0 | 7.9 ± 1.8 | 10.8 ± 1.6 | 17.65 ± 2.70 |
| B | 1.9 ± 1.2 | 3.1 ± 1.1 | 9.2 ± 1.4 | 12.1 ± 1.9 | 20.4 ± 2.4 |

TABLE 6
Enhancement of cytotoxicity (%) for gemcitabine-loaded unmodified
apatite-based matrix (A) and pharmaceutical composition comprising
gemcitabine (B) in 4T1 cell line, in an increasing concentration.

| | 100 pM | 1 nm | 10 nM | 100 nM | 1 uM |
|---|---|---|---|---|---|
| A | 1.0 ± 1.8 | 3.2 ± 1.2 | 3.5 ± 1.7 | 5.7 ± 2.0 | 10.4 ± 1.3 |
| B | 1.5 ± 1.9 | 3.8 ± 1.1 | 3.60 ± 1.15 | 8.5 ± 1.6 | 11.7 ± 1.5 |

TABLE 7
Enhancement of cytotoxicity (%) for anastrozole-loaded unmodified
apatite-based matrix (A) and pharmaceutical composition comprising
anastrozole (B) in MCF 7 cell line, in an increasing concentration.

| | 100 pM | 1 nm | 10 nM | 100 nM | 1 uM |
|---|---|---|---|---|---|
| A | 1.1 ± 2.5 | 1.34 ± 2.0 | 4.2 ± 1.4 | 5.04 ± 2.20 | 0.8 ± 1.7 |
| B | 1.8 ± 1.4 | 3.7 ± 2.3 | 6.1 ± 2.4 | 7.2 ± 2.3 | 3.0 ± 1.9 |

TABLE 8
Enhancement of cytotoxicity (%) for anastrozole-loaded unmodified
apatite-based matrix (A) and pharmaceutical composition comprising
anastrozole (B) in 4T1 cell line, in an increasing concentration.

| | 100 pM | 1 nm | 10 nM | 100 nM | 1 uM |
|---|---|---|---|---|---|
| A | 1.6 ± 1.5 | 2.0 ± 2.4 | 6.3 ± 2.0 | 11.5 ± 2.7 | 8.7 ± 3.2 |
| B | 2.2 ± 2.0 | 4.7 ± 1.6 | 8.80 ± 2.25 | 18.4 ± 2.1 | 10.60 ± 3.76 |

### Example 4

### Evaluation of Antitumor Activity Using Tumour Model Mice

(3) ESR1 and BCL-2 siRNAs

Tumour outgrowth of mice were intravenously treated with: i) apatite-based matrix as control (CA-treated); ii) apatite-based matrix incorporated with ESR1 siRNA (CA+ESR1); iii) apatite-based matrix incorporated with BCL-2 siRNA (CA+BCL-2); iv) apatite-based matrix incorporated with ESR1 and BCL-2 siRNAs (CA+ESR1+BCL-2); and v) without treatment (untreated) respectively using a 4T1 induced breast tumour mouse model.

Mice were administered twice (three days apart) with 100 μL of aqueous solution containing no treatment, CA-treated, CA+ESR1, CA+BCL-2 and CA+ESR1+BCL-2 complexes respectively. Apatite-siRNA complexes were formed by mixing 50 mM of a particular siRNA along with different salts (Ca²⁺, Fe²⁺/Fe²⁺, Mg²⁺, NaCl, bicarbonate and inorganic phosphate) and glucose in 100 μL of an aqueous solution and incubating the mixture at 37° C. for 30 mins. Measurement on tumour outgrowth volume of mice (mm³) were taken at day 8, 10, 12, 14, 16, 18, 22 and 24.

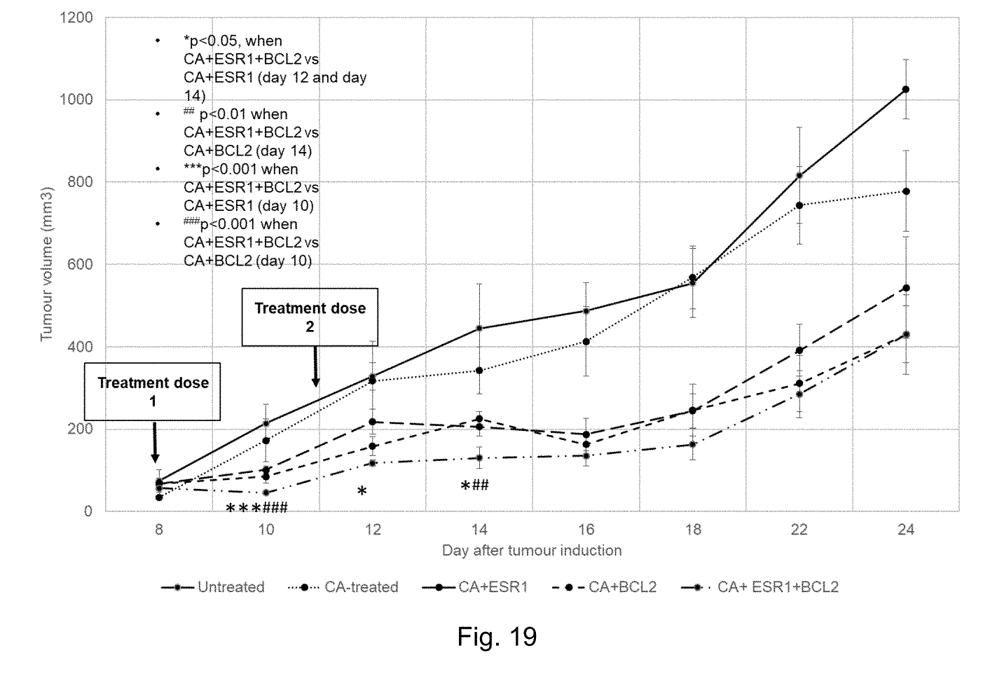

The result is shown in FIG. 19. Six mice per group were used and data were represented as mean±SD. Values were significant with p<0.05 (*) compared to the control group.

As shown in FIG. 19, intravenous delivery of apatite-based matrix complexes of either anti-ESR1 or anti-BCL-2 siRNA significantly reduced the tumour load in a consecutive manner from day 10 to day 24, confirming the vital role of ESRI as well as BCL-2 in progression (survival and/or proliferation) of 4T1 mammary carcinoma. Moreover, combined delivery of the siRNAs targeting both ESR1 and BCL-2 siRNAs showed a trend of further declining the tumour mass, particularly at the earlier stage of the experimental period.

(4) ESR1, ERBB2 and EGFR siRNAs

Tumour outgrowth of mice were intravenously treated with: i) apatite-based matrix as control (CA-treated); ii) apatite-based matrix incorporated with ERBB2 siRNA (CA+ERBB2); iii) apatite-based matrix incorporated with ESR1 siRNA (CA+ESR1); iv) apatite-based matrix incorporated with EGFR siRNA (CA+EGFR); v) apatite-based matrix incorporated with ESR1, ERBB2 and EGFR siRNAs (CA+ESR1+ERBB2+EGFR); and vi) without treatment (untreated) respectively using a 4T1 induced breast tumour mouse model.

The following procedure was carried out as described above. Measurement on tumour outgrowth volume of mice (mm³) were taken at day 8, 10, 12, 14, 16, 18, 22 and 24.

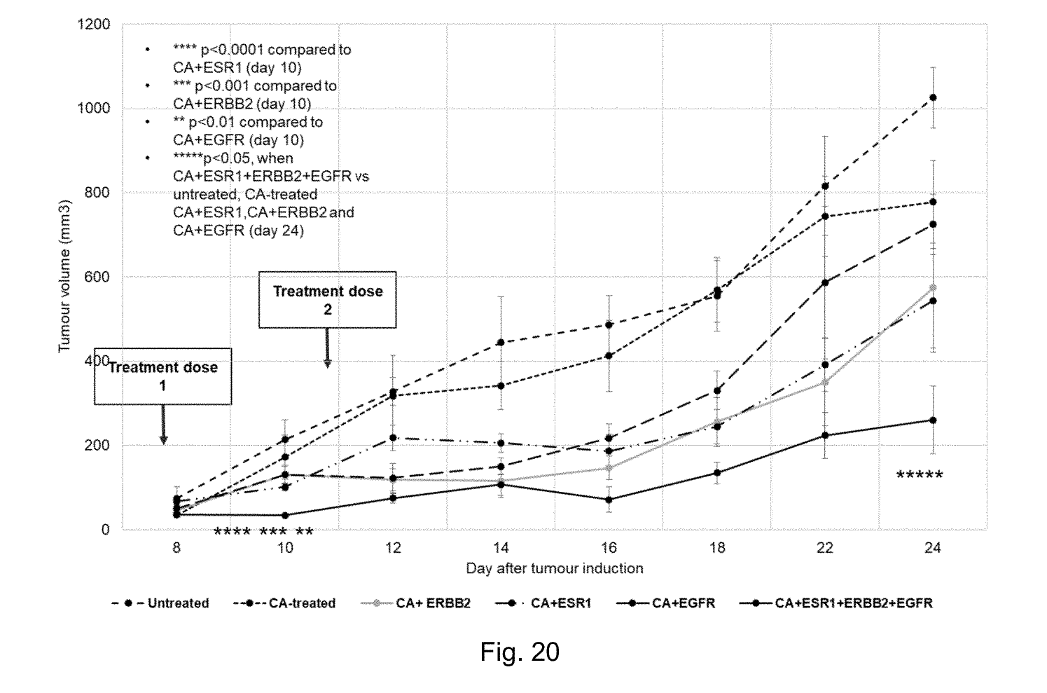

The result is indicated in FIG. 20. Six mice per group were used and data were represented as mean±SD. Values were significant with p<0.05 (*) compared to the control group.

CA+ESR1+ERBB2+EGFR complex demonstrated potent cytotoxic effect with suppression of expression and activation of MAPK and PI-3 kinase pathways in MCF-7 cells and more remarkably in 4T1 cells, with exception in MDA-MB-231 cells (not shown). Treatment of 4T1 tumours with the single siRNAs targeting either EGFR or HER2 resulted in similar reduction in tumour volume as with ESR1 siRNA, demonstrating the active involvement of these three growth factors in 4T1 tumour growth and/or survival. In addition, combined delivery of the siRNAs against all these three growth factor receptors led to a further decline in tumour mass over the entire period except day 14 as indicated in FIG. 20, suggesting that simultaneous targeting of these receptors has huge implication for therapeutic intervention in breast cancer.

(8) ROS1 and SHC siRNA

Tumour outgrowth of mice were intravenously treated with i) apatite-based matrix as control (CA-treated); ii) apatite-based matrix incorporated with ROS1 siRNA (CA+ROS1); iii) apatite-based matrix incorporated with SHC1 siRNA (CA+SHC1); iv) apatite-based matrix incorporated with ROS1 and SHC1 siRNAs (CA+ROS1+SHC1); and v) without treatment (untreated) respectively using a 4T1 induced breast tumour mouse model.

The following procedure was carried out as described above. Measurement on tumour outgrowth volume of mice (mm³) were taken at day 8, 10, 12, 14, 16, 18 and 20.

The result is indicated in FIG. 21. Data were represented as mean±SD. Values were significant with p<0.05 (**) compared to the control group.

As shown in FIG. 21, individual and combined (synergistic) effects in cancer cell killing were observed following delivery of the siRNAs against ROS1 and SHC1 both in vitro and in vivo.

(9) ROCK2, CAMK4, NFATC4 and RYR3 siRNAs Tumour outgrowth of mice were intravenously treated with i) apatite-based matrix as control (CA-treated); ii) apatite-based matrix incorporated with ROCK2 siRNA (CA+ROCK2); iii) apatite-based matrix incorporated with CAMK4 siRNA (CA+CAMK4); iv) apatite-based matrix incorporated with NFATC4 siRNA (CA+NFATC4); v) apatite-based matrix incorporated with RYR3 siRNAs (CA+RYR3); vi) apatite-based matrix incorporated with ROCK2, CAMK4, NFATC4 and RYR3 siRNAs (CA+ROCK2+CAMK4+NFATC4+RYR3); and vii) without treatment (untreated) respectively using a 4T1 induced breast tumour mouse model.

The following procedure was carried out as described above. Measurement on tumour outgrowth volume of mice (mm3) were taken at day 10, 13, 16, 19 and 22.

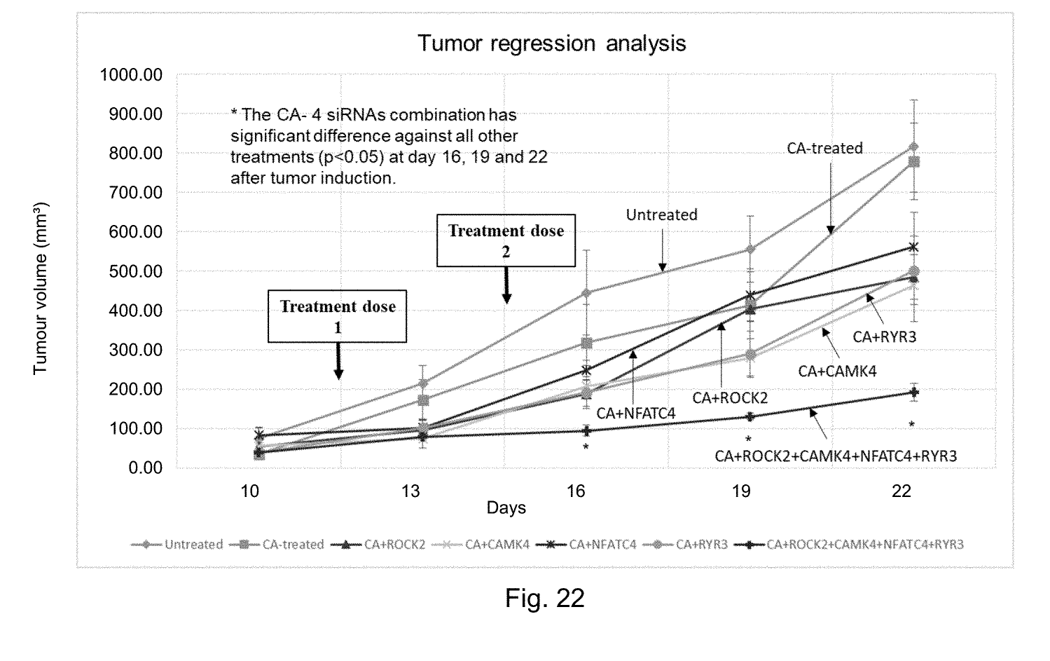

The result is indicated in FIG. 22. Data were represented as mean±SD. Values were significant with p<0.05 (*) compared to the control group.

As shown in FIG. 22, there were significant cytotoxicity and tumour regression effects observed similarly as in FIG. 21 by down-regulating the expression of ROCK2, CAMK4, NFATC4 and RYR3 following in vitro and in vivo delivery of the respective siRNAs (individually or in combination) using the apatite-based matrix.

Further, there are studies still on-going to employ the pharmaceutical composition for delivering genes of caspase 2, caspase 3, caspase 7, caspase 8, BRCA 1, BRCA 2, PTEN, p21, and p53, either individually or in combination, into the breast cancer cells in order to achieve significant toxicity.

(10) Gemcitabine

Tumour regression study following intravenous delivery of i) no treatment; ii) apatite-based matrix (NP); iii) free gemcitabine; iv) apatite-based matrix with gemcitabine; and v) pharmaceutical composition comprising gemcitabine respectively into 4T1-induced breast tumours in mice was carried out to evaluate the antitumor effect using the pharmaceutical composition.

4T1 cells were inoculated subcutaneously on the mammary pad of mice. Tumour-bearing mice were treated intravenously through tail vein injection with 100 μL solution containing i) no treatment; ii) apatite-based matrix; iii) free gemcitabine (0.34 mg/Kg); iv) unmodified apatite-based matrix formed with 0.34 mg gemcitabine/Kg; and v) pharmaceutical composition with 0.34 mg gemcitabine/Kg respectively, when the tumour volume reached to 13.20±2.51 mm³. The injections were intravenously administered twice within an interval of 3 days to a 4T1 cancer cells-induced syngeneic mouse model of breast cancer. Measurements on tumour volume at day 8, 10, 12, 14, 16, 18, 20 and 22 were taken. The concentration of detected drug in tumour were also measured, followed by observation on bio-distribution of drugs in each organ. Six mice per group were used and data were represented as mean±SD of tumour volume.

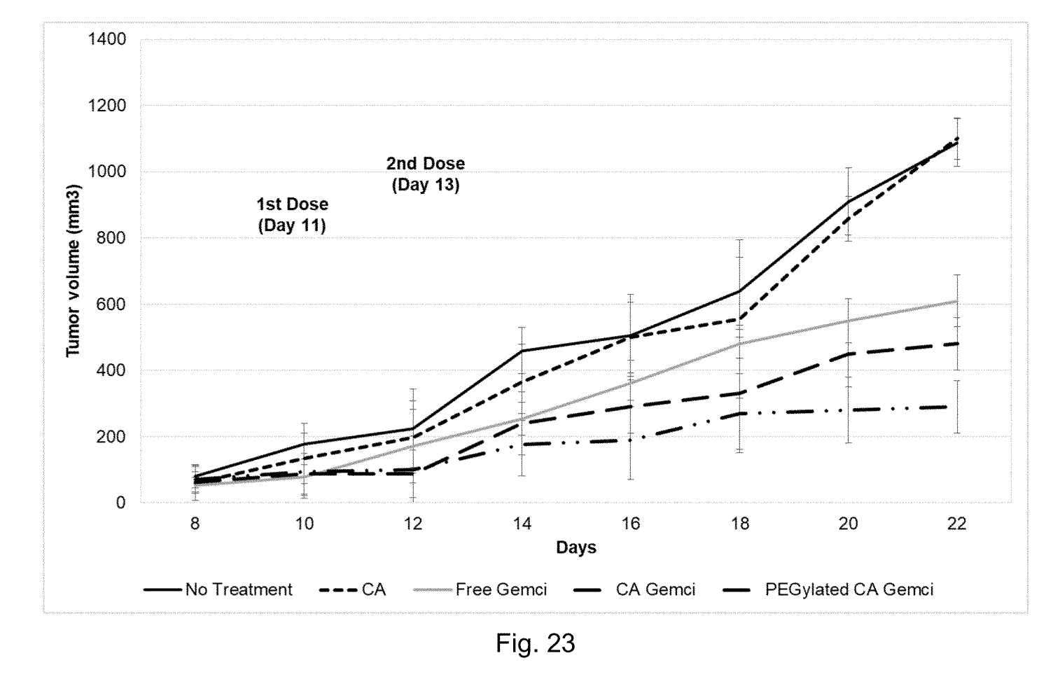

As shown in FIG. 23, compared to free gemcitabine (0.5 mg/Kg of a mouse), unmodified apatite-based matrix loaded with the same amount of the drug led to a significant reduction in tumour volume, while the surface-modified ones dramatically regressed the tumour growth, indicating that surface modification might confer the favourable pharmacokinetics of the pharmaceutical composition with higher accumulation and uptake by the tumour.

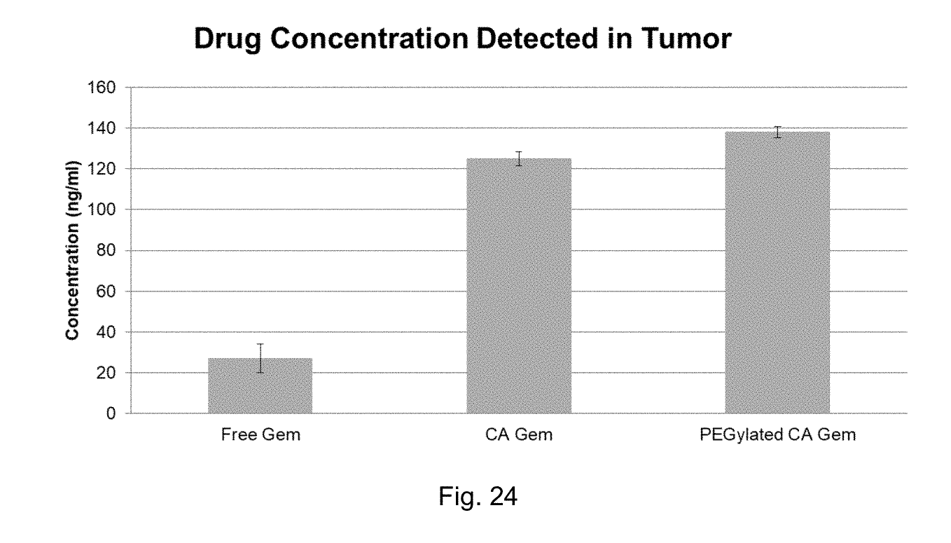

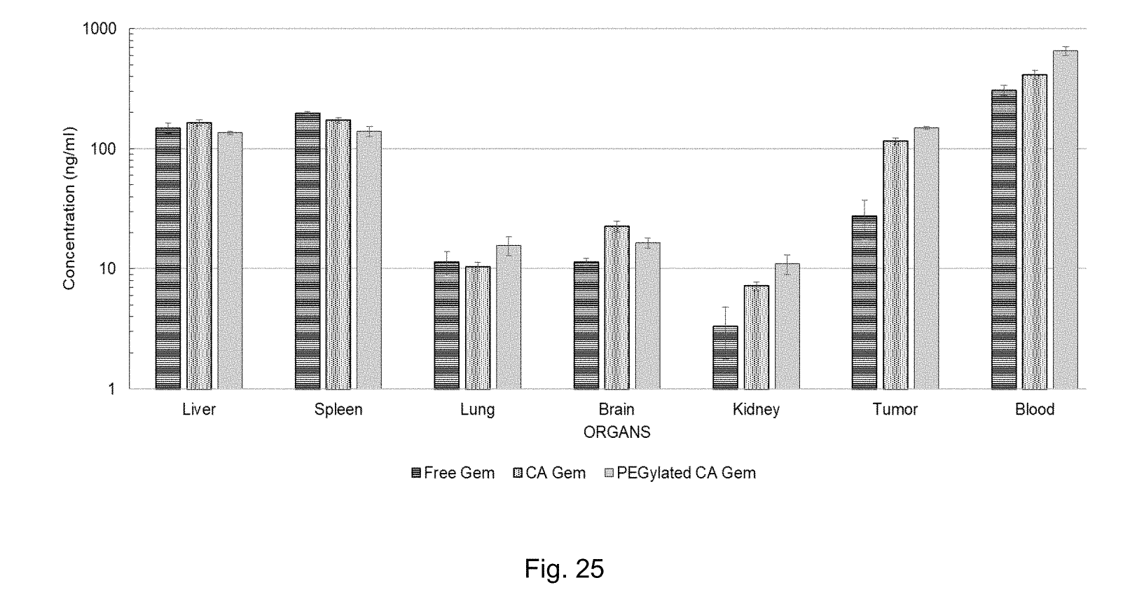

As shown in FIG. 24 and Table 9, following intravenous delivery, compared to free gemcitabine (50 mg/Kg of a mouse), unmodified apatite-based matrix loaded with the same amount of the drug led to more than 5-fold increase in drug accumulation in the tumour, while the surface-modified ones caused further increase in the tumour uptake of the drug. The results suggest that apatite-based particles in the pharmaceutical composition enhanced significant tumour accumulation of the bound drug by preventing homogeneous tissue distribution of the drug, whereas PEGylation in the pharmaceutical composition showed a further increase in the uptake, probably by preventing opsonisation of the particles by macrophages. The analysis of drug accumulation in other organs is shown in FIG. 25.

TABLE 9
Concentration of detected drug accumulated in tumour following
intravenous treatment as in Example 4 (5).

| | Detected Drug | Standard |
|---|---|---|
| Treatment | Concentration (ng/ml) | Deviation |
| Free Gemcitabine | 27,03333 | 6,990153 |
| CA-Gemcitabine | 125,9433 | 3,444798 |
| PEGylated CA-Gemcitabine | 138,0367 | 2,64606 |

### Example 5

### Selective Tumour Accumulation Activity Using Tumour Model Mice

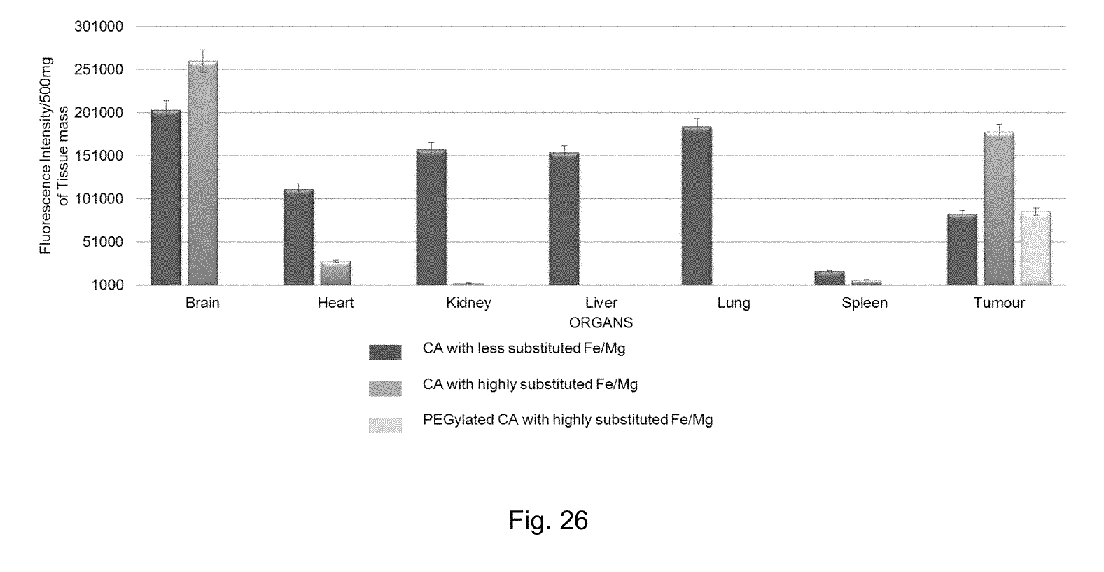

Further evaluation on the selective accumulation of the pharmaceutical composition in tumours and other organs is shown in FIG. 26 via intravenous injection of pharmaceutical composition containing fluorescence siRNA through mouse tail vein. The pharmaceutical composition was fabricated using higher amount of Fe²⁺/Fe³⁺ and Mg²⁺ ions as well as higher amount of surface modifying agent, particularly biotinylated PEG.

With the higher amount of Fe²⁺/Fe³⁺ and Mg²⁺ ions as well as the surface modifying agent, the pharmaceutical composition showed to be more selective accumulation activity in the tumour regions as compared to other organs, as indicated by FIG. 26.

### Example 6

### Addition of Carboxyl Group-Containing Biological Molecules in Production of Apatite-Based Matrix

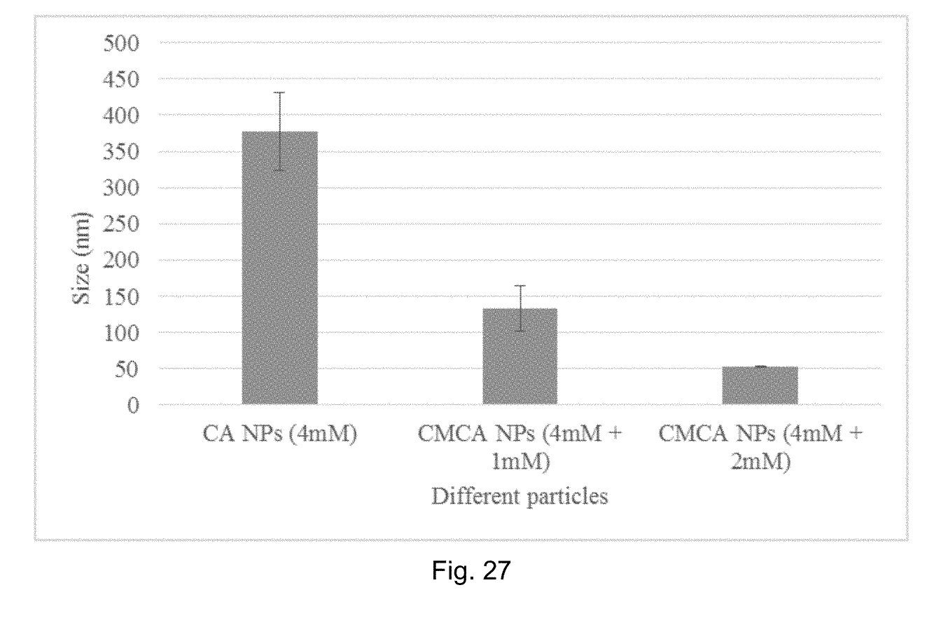

Role of carboxyl group-containing biological molecules in production of apatite-based matrix was tested by adding the biological molecules, particularly citrate into the mixture as prepared previously containing all the necessary ions before addition of the surface modifying agent, thereby forming the end product which is citrate-incorporated apatite-based matrix (denoted briefly as CMCA NP). Furthermore, CMCA was fabricated using two different concentrations of citrate at 1 mM and 2 mM, while the concentration of Ca²⁺ was prepared at 4 mM while other components were fixed as prepared as above. The average size of the CMCA was compared with free apatite-based matrix (denoted briefly as CA NP) and the result was tabulated in FIG. 27. The average size of CA NPs was around 416 nm, while the average sizes of CMCA NP which were formulated with 1 mM and 2 mM concentration of sodium citrate were approximately 163 nm and 53 nm, respectively.

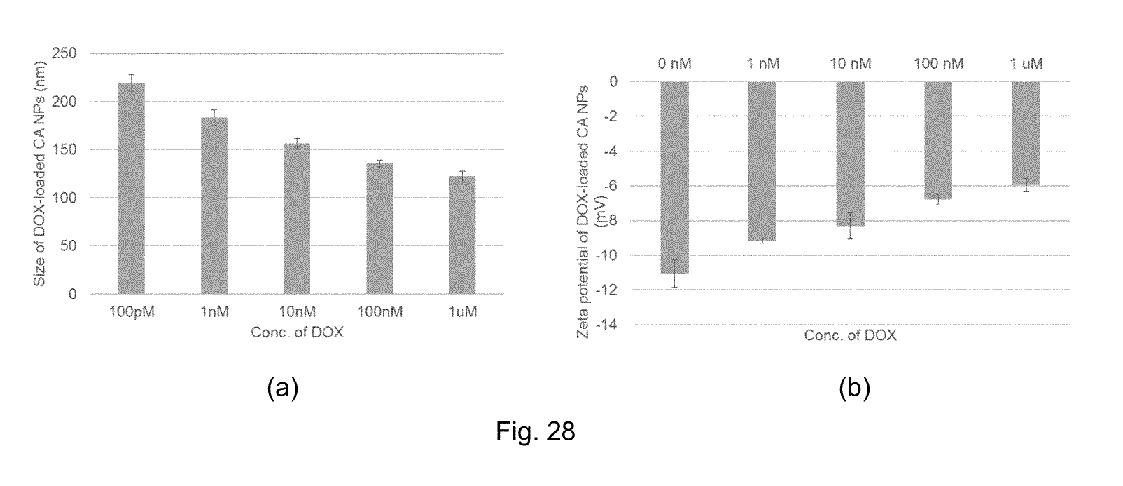

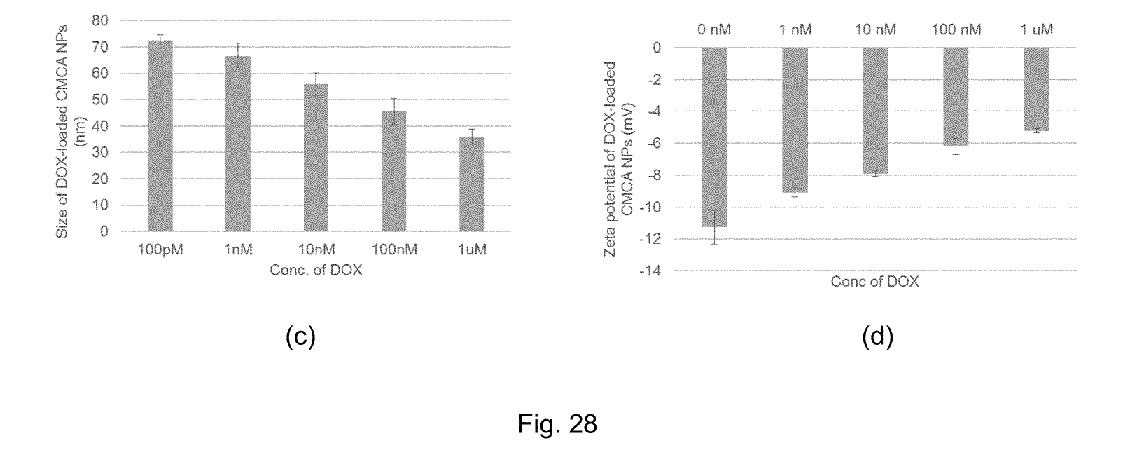

Further, the interaction between CMCA NP and the drug i.e. doxorubicin (denoted briefly as Dox) was studied by comparing the size and binding affinity of CA NP and CMCA NP that were loaded with Dox. The positively charged DOX might bind electrostatically with negatively charged domains (rich in bicarbonate, phosphate or citrate) on CMCA NP and CA NP, causing the net charge of the DOX-loaded CMCA NPs and CA NPs more electropositive than that of the free NPs, as shown in FIGS. 28 (b) and (d). A higher binding affinity of CMCA NPs and CA NPs towards the drugs dramatically reduced the size of drug-particle complexes, as indicated in FIGS. 28 (a) and (c) which are more suitable for cellular uptake. In addition, the evaluation on cellular uptake of Dox-loaded CA NPs and CMCA NPs was performed after 1 hour and 4 hours of treatment. The results were tabulated and shown in Table 10 and 11.

TABLE 10
Percentage (%) of Cellular uptake for DOX 1 μM, DOX (1 μM)-CA NPs
and DOX (1 μM)-CMCA NPs after 1 hour and 4 hours of treatment.

| | % Cellular uptake 1 hour of | % Cellular uptake |
|---|---|---|
| Formulation | treatment | 4 hours of treatment |
| DOX | 22.4% ± 1.73 | 31.71% ± 5.92 |
| DOX-CA | 32.69% ± 0.38 | 41.09% ± 0.79 |
| DOX-CMCA | 36.45% ± 0.72 | 48.93% ± 1.32 |

TABLE 11
Cellular uptake for DOX 5 μM, DOX (5 μM)-CA NPs and DOX (5 μM)-
CMCA NPs after 1 hr and 4 hr of treatment.

| | % Cellular uptake 1 hour of | % Cellular uptake 4 hours |
|---|---|---|
| Formulation | treatment | of treatment |
| DOX | 24.75% ± 0.14 | 32.07% ± 0.82 |
| DOX-CA | 38.26% ± 0.07 | 41.14% ± 0.26 |
| DOX-CMCA | 40.64% ± 0.12 | 47.41% ± 0.11 |

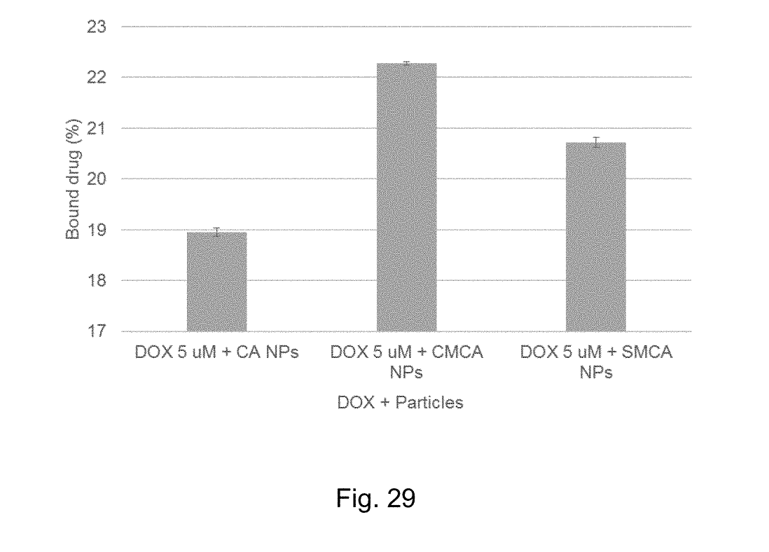

Besides that, further analysis and evaluation were conducted to determine the role of the carboxyl group-containing biological molecules. Another biological molecule i.e. succinate was used to be incorporated into CA NPs, thereby forming succinate-incorporated apatite-based matrix (denoted briefly as SMCA NP). The efficiency of drug binding to the apatite-based matrix was further evaluated by determining the drug binding affinity towards Dox-loaded CA NPs, Dox-loaded CMCA NPs and Dox-loaded SMCA NPs. The result was tabulated in FIG. 29. As shown in FIG. 29, Dox possessed 18.95%, 20.72% and 22.27% binding affinity for CA, SMCA and CMCA NPs at 5 μM concentration, respectively.

Furthermore, similar in vitro chemosensitivity assay has been conducted for Dox-loaded CA NPs, Dox-loaded CMCA NPs and Dox-loaded SMCA NPs respectively. The result has been tabulated in the Table 12-14.

TABLE 12
Enhancement of cytotoxicity (%) for DOX-loaded CMCA NPs.

| Concentration of DOX | MCF-7 | 4T1 |
|---|---|---|
| 1 pM | 13.35 ± 1.92 | 4.14 ± 1.4 |
| 10 pM | 16.90 ± 1.34 | 12.42 ± 1.62 |
| 100 pM | 10.85 ± 1.87 | 13.45 ± 2.27 |
| 1 nM | 15.66 ± 2.69 | 17.72 ± 2.58 |
| 10 nM | 15.12 ± 3.25 | 15.78 ± 4.32 |
| 100 nM | 20.99 ± 1.93 | 19.40 ± 1.34 |
| 1 μM | 25.62 ± 0.82 | 11.77 ± 1.25 |

TABLE 13
Enhancement of cytotoxicity (%) for DOX-loaded CA NPs.

| Concentration of DOX | MCF-7 | 4T1 |
|---|---|---|
| 1 pM | 1.78 ± 2.52 | 0.91 ± 2.58 |
| 10 pM | 2.67 ± 3.5 | 7.37 ± 0.22 |
| 100 pM | 1.60 ± 2.97 | 3.62 ± 3.01 |
| 1 nM | 1.42 ± 1.92 | 1.16 ± 1.24 |
| 10 nM | 1.94 ± 2.7 | 3.23 ± 2.20 |
| 100 nM | 1.11 ± 2.93 | 3.62 ± 4.25 |
| 1 μM | 11.03 ± 1.34 | 5.04 ± 5.1 |

TABLE 14
Enhancement of cytotoxicity (%) for DOX-loaded SMCA NPs.

| Concentration of DOX | MCF-7 | 4T1 |
|---|---|---|
| 1 pM | 2.89 ± 6.68 | 2.99 ± 6.76 |
| 10 pM | 17.33 ± 2.24 | 7.49 ± 2.79 |
| 100 pM | 7.64 ± 2.93 | 10.64 ± 1.04 |
| 1 nM | 7.52 ± 0.54 | 11.64 ± 1.94 |
| 10 nM | 6.25 ± 2.56 | 16.93 ± 1.41 |
| 100 nM | 8.55 ± 2.11 | 20.88 ± 7.48 |
| 1 μM | 7.60 ± 2.92 | 22.53 ± 1.29 |

In conclusion, carboxyl group-containing biological molecules, such as citrate, succinate, pyruvate, lactate, alpha-ketoglutarate, oxaloacetate, fumarate and malate electrostatically interacts with the apatite-based nanoparticles, thereby changing or stabilizing the particle size, increasing the drug binding into the particles, promoting more cellular uptake of the drug and consequentially, enhancing cytotoxicity of the drug as part of future cancer treatment. For example, binding of citrate led to a dramatic decrease in diameter (size) of original Fe/Mg-substituted CA NPs in a dose-dependent manner of citrate and the resultant CMCA NPs exhibited the highest (31.38%) binding affinity for doxorubicin (as measured using the interaction efficiency (%) formula described above) and promoted rapid cellular uptake of the drug, leading to the half-maximal inhibitory concentration 1000 times less than that of the free drug in MCF-7 cells. Hence, CMCA NPs with greater surface area enhance cytotoxicity in different breast cancer cells by enabling higher loading and more efficient cellular uptake of the drug.

On the other hand, cyclophosphamide, another anti-cancer drugs has showed similar effects as Dox (data not showing here). Therefore, all hydrophilic and hydrophobic anti-cancer drugs can be used to be encapsulated into CMCA or SMCA nanoparticles with the above desirable advantages, such as drastic reduction in drug/particle complexes, more cellular uptake and enhanced cytotoxicity. CMCA or SMCA nanoparticles may also be further coated with the surface modifying agent i.e. biotinylated PEG. As the particles may have smaller size and may further subject to surface modification, these particles are highly expected to show better pharmacokinetic (bio-distribution) profiles in animal models and patients, with enhanced tumour accumulation and minimal uptake by other organs

The above-mentioned pharmaceutical composition overcomes the problems and shortcomings of the existing pharmaceutical composition comprising inorganic and organic components and provides a number of advantages over them. The pharmaceutical composition comprising an inorganic apatite-based matrix and an organic surface modifying agent, in which the inorganic apatite-based matrix is important in regulating the size of the pharmaceutical composition, while the surface modifying agent plays significant role in improving the bio-distribution profile of the pharmaceutical composition. The invention aids in facilitate targeting on specific cell-surface receptors to eliminate off-target effects and eventually enhance therapeutic efficacy. Also, the invention may confer a favourable pharmacokinetics and efficient release of drugs in the target cells through surface modification and pH sensitivity control on the pharmaceutical composition. Further, the invention has the capability of overcoming the limitation of poor complexation with multiple hydrophobic and hydrophilic drugs that encountered by the existing arts. Moreover, the invention is able to eliminate particles aggregation possibly caused by ionic and hydrophobic interactions among the apatite-based matrix, solvent and drug molecules.

The exemplary implementation described above is illustrated with specific shapes, dimensions, and other characteristics, but the scope of the invention includes various other shapes, dimensions, and characteristics. Also, the pharmaceutical composition as described above could be fabricated in various other ways and could include various other materials, including various other ions, protein, polymers etc.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claim.

1. A pharmaceutical composition comprising:
a pharmaceutically active substance;
an apatite-based matrix; and
a surface modifying agent;
characterized in that
the apatite-based matrix comprising calcium ion, phosphate ion, hydrogen carbonate ion, magnesium ion and iron ion;
the surface modifying agent comprising a protein, a polymer or a combination thereof.

2. The pharmaceutical composition as claimed in claim 1, characterized in that the apatitie-based matrix further comprises at least one ion selected from strontium ion, fluoride ion and barium ion, or at least one carboxylate group-containing monomer selected from citrate, succinate, pyruvate, lactate, alpha-ketoglutarate and oxaloacetate, or any combination thereof.

3. The pharmaceutical composition as claimed in claim 1, characterized in that the protein is streptavidin, transferrin, fibronectin, collagen, albumin, lactoferrin, asialofetuin, lipoprotein or proteoglycan.

4. The pharmaceutical composition as claimed in claim 1, characterized in that the polymer is polyethylene glycol (PEG).

5. The pharmaceutical composition as claimed in claim 4, characterized in that each PEG is associated with a biotin moiety.

6. The pharmaceutical composition as claimed in claim 1, characterized in that the size of the pharmaceutical composition is 5-999 nanometer.

7. The pharmaceutical composition as claimed in claim 1, characterized in that the pharmaceutically active substance is selected from the group consisting of drug, protein, nucleic acid and any combination thereof.

8. The pharmaceutical composition as claimed in claim 7, characterized in that the drug is an anti-tumor agent.

9. The pharmaceutical composition as claimed in claim 8, characterized in that the anti-tumor agent is selected from the group comprising of antimetabolites, alkylating agents and antibiotics.

10. The pharmaceutical composition as claimed in claim 7, characterized in that the nucleic acid is deoxyribonucleic acid (DNA), ribonucleic acid (RNA), oligonucleotide or polynucleotide.

11. The pharmaceutical composition as claimed in claim 10, characterized in that the ribonucleic acid is siRNA, miRNA or antisense of RNA.

12. A method for producing the pharmaceutical composition (200) comprising the steps of:
preparing a first mixture containing the pharmaceutically active substance and the apatite-based matrix (202);
subjecting the first mixture to a first incubation (204);
adding a surface modifying agent into the first mixture to form a second mixture (206); and
subjecting the second mixture to a second incubation (208) to form the pharmaceutical composition.

13. The method for producing the pharmaceutical composition (200) as claimed in claim 12, characterized in that the first mixture is further added with at least one ion selected from strontium ion, fluoride ion and barium ion, or at least one carboxyl group-containing molecules selected from citrate, succinate, pyruvate, lactate, alpha-ketoglutarate, oxaloacetate, fumarate and malate, or any combination thereof before the first incubation step (204).

14. The method for producing the pharmaceutical composition (200) as claimed in claim 12, characterized in that the first mixture is further added with a protein-based surface modifying agent before the first incubation step (204).

15. The method for producing the pharmaceutical composition (200) as claimed in claim 12, characterized in that the apatite-based matrix is prepared by the steps comprising of:
preparing a first solution that contains calcium ion;
adding the first solution into a second solution that contains phosphate ions, hydrogen carbonate ion, magnesium ion and iron ion.

16. The method for producing the pharmaceutical composition (200) as claimed in claim 12, characterized in that the apatite-based matrix is prepared by the steps comprising of:
preparing a first solution that contains phosphate ion;
adding the first solution into a second solution that contains calcium ion, hydrogen carbonate ion, magnesium ion and iron ion.

17. The method for producing the pharmaceutical composition (200) as claimed in claim 15 or 16, characterized in that the second solution further comprising sodium chloride and glucose.

18. The method for producing the pharmaceutical composition (200) as claimed in claim 17, characterized in that the concentration of sodium chloride is in a range of 10-1000 millimolar of the second solution.

19. The method for producing the pharmaceutical composition (200) as claimed in claim 17, characterized in that the concentration of glucose is in a range of 10-1000 millimolar of the second solution.

20. The method for producing the pharmaceutical composition (200) as claimed in claim 15 or 16, characterized in that the calcium ion concentration is in a range of 1-100 millimolar.

21. The method for producing the pharmaceutical composition (200) as claimed in claim 15 or 16, characterized in that the phosphate ion concentration is in a range of 0.1-100 millimolar.

22. The method for producing the pharmaceutical composition (200) as claimed in claim 15 or 16, characterized in that the hydrogen carbonate ion concentration is in a range of 10-100 millimolar.

23. The method for producing the pharmaceutical composition (200) as claimed in claim 15 or 16, characterized in that the magnesium ion concentration is in a range of 1-100 millimolar.

24. The method for producing the pharmaceutical composition (200) as claimed in claim 15 or 16, characterized in that the iron ion concentration is in a range of 1-100 millimolar.

25. The method for producing the pharmaceutical composition (200) as claimed in claim 12, characterized in that the first mixture has a pH of 6.0-8.0.

26. The method for producing the pharmaceutical composition (200) as claimed in claim 12, characterized in that each incubation is carried out at a temperature in a range of 25° C.-65° C.

27. The method for producing the pharmaceutical composition (200) as claimed in claim 12, characterized in that the pharmaceutical composition is dispersed in a pharmacologically acceptable solvent when in use.

28. The method for producing the pharmaceutical composition (200) as claimed in claim 27, characterized in that the pharmacologically acceptable solvent is a buffered cell culture medium solution or saline solution.

29. The method for producing the pharmaceutical composition (200) as claimed in claim 12, characterized in that the pharmaceutical composition is subjected to lyophilisation to obtain a powder form.

30. The method for producing the pharmaceutical composition (200) as claimed in claim 12, characterized in that the pharmaceutical composition is subjected to high pressure condensation to obtain a solid dosage form.
